(19) United States
(12) Patent Application Publication
(10) Pub. No.: US 2007/0011772 A1
(43) Pub. Date: Jan. 11, 2007

(54) Polynucleotides encoding a beta-glucosidase and uses thereof

(75) Inventors: Volker Lipka, Koln (DE); Dierk Scheel, Halle (DE); Paul Schulze-Lefert, Koln (DE); Sabine Rosahl, Halle (DE); Jorn Landtag, Halle (DE)

Correspondence Address: NOVAK DRUCE DELUCA & QUIGG, LLP, 1300 EYE STREET NW, SUITE 400 EAST TOWER, WASHINGTON, DC, 20005, US

(73) Assignee: Max-Planck-Gesellschaft Zur Forderung Der Wissenschaften E.V., Berlin (DE)

(21) Appl. No.: 10/506,670

(22) PCT Filed: Mar. 6, 2003
(86) PCT No.: PCT/EP03/02315

§ 371 Date: Jul. 8, 2005

(30) Foreign Application Priority Data
Mar. 6, 2002 (EP) 02004400.4

(51) Int. Cl. A01H 5/00; C12Q 1/68; G06F 19/00; C07H 21/04; C12N 15/82; A01H 1/00; C12N 5/04; C07K 14/415; C12N 9/48

(52) U.S. Cl. 800/279; 530/370; 435/6; 435/419; 435/468; 536/23.6; 702/19; 435/212

(57) ABSTRACT

Disclosed are polynucleotides that encode a beta-glucosidase belonging to the plant protein class of family 1 glycoside hydrolases. Furthermore described are recombinant nucleic molecules and vectors containing these polynucleotides and host cells, preferably plant cells that are genetically engineered with these polynucleotides. In addition, transgenic plants are disclosed which show an increased activity of the beta-glucosidase described, preferably leading to an improved resistance against plant pathogens. In another aspect, the present application discloses screening methods for identifying a substrate for said betaglucosidase and methods for the production of plant protection compositions from such substrates or hydrolysis products thereof. Furthermore, methods and uses are disclosed which apply the polynucleotides encoding the beta-glucosidase described in order to improve resistance in plants against plant pathogens.

The present invention relates to polynucleotides that encode a beta-glucosidase belonging to the plant protein class of family 1 glycoside hydrolases. The present invention furthermore relates to recombinant nucleic molecules and vectors containing these polynucleotides and to host cells, preferably plant cells, that are genetically engineered with these polynucleotides. In addition, the present invention relates to transgenic plants which show an increased activity of the beta-glucosidase of the invention, preferably leading to an improved resistance against plant pathogens. In another aspect, the present invention relates to screening methods for identifying a substrate for said beta-glucosidase and to the production of plant protection compositions from such substrates or hydrolysis products thereof. Furthermore, the present invention relates to methods and uses which apply the polynucleotides encoding the beta-glucosidase of the invention in order to improve resistance in plants against plant pathogens.

In plants, a major form of resistance to disease caused by microbial pathogens is by expression of complementary gene pairs in the plant and pathogen, known respectively as resistance (R) and avirulence (avr) genes (Dangl and Jones, 2001). Direct or indirect interaction of their products triggers rapid cellular reprogramming that prevents pathogen colonization of the plant. Failure to express either one component results in disease susceptibility. The predominant class of plant R gene cloned from a diverse range of species encodes cytoplasmic proteins with the same modular structure as Nod proteins involved in animal innate immune responses (Dangl and Jones, 2001; Fluhr, 2001).

Another form of plant defense is nonhost resistance, which describes the fact that an entire plant species is normally immune to a specific parasite (Heath, 2000). Because most plant species are immune to the majority of potentially pathogenic microorganisms, nonhost resistance most probably constitutes a major factor of resistance in the field. Also, nonhost resistance is known to be considerably more durable in nature compared to strain-specific R gene dependent resistance. Despite its durable nature, nonhost resistance can depend on the recognition of a single pathogen molecule (e.g. recognition of the Phytophthora infestans Inf1 protein by Nicotiana benthamiana; Kamoun et al., 1998). Generally, however, nonhost resistance is at present poorly understood at the molecular level.

The Erysiphales, commonly known as the powdery mildews, are obligate biotrophic plant pathogenic fungi, belonging to the phylum Ascomycota. The fungi are the causal agent of the widespread powdery mildew disease. Powdery mildews were reported to infect at least 9,838 angiosperm plant species representing 1,617 genera (Amano, 1986). Remarkable is the host range variation of powdery mildew species. For example, the barley powdery mildew fungus (Blumeria graminis f sp hordei; Bgh) successfully colonizes barley but fails to infect even close relatives including wheat, rye, and oat. In contrast, Erysiphe cichoracearum has a much broader host range and infects several dicot plant species that belong to different families (e.g. Arabidopsis, squash, and tobacco). It is possible that host range is not determined by the absence/presence of static or inducible defense responses in a plant species but by lack of compatibility factors or absence of virulence factors in a powdery mildew species.

Yet, the classic approaches of plant breeding focus on trying to introduce resistance traits from land races into commercially used plant varieties. This refers to genes conferring race-specific resistance as well as to non-host resistance.

Since the advent of genetic engineering, many approaches have been made aiming at improving pest resistance in plants by producing transgenic plants that express recombinant gene constructs. For instance, it was tried to introduce pathogenesis-related protein genes into the plant genome in order to enhance the plant response to pathogen attack. However, for many pests, a sweeping success is still outstanding since one had to learn that plant-pathogen interactions are much more complex than previously imagined. Therefore, the introduction of one transgene often proved not to modify the complex regulatory network triggered upon infection in such a way that plant resistance was significantly improved. Thus, there is still a need to find effective tools for improving non-host resistance in plants.

Thus, the technical problem underlying the present invention is to provide means and methods that allow it to improve plants by establishing or enhancing a broad, preferably non-host resistance against plant pathogens.

This technical problem is solved by the provision of the embodiments as characterized in the claims.

Accordingly, the present invention relates to polynucleotides selected from the group consisting of
(a) polynucleotides comprising a nucleotide sequence encoding a polypeptide with the amino acid sequence of SEQ ID NO:2;
(b) polynucleotides comprising the nucleotide sequence shown in SEQ ID NO:1;
(c) polynucleotides comprising a nucleotide sequence encoding a fragment of the polypeptide encoded by a polynucleotide of (a) or (b), wherein said nucleotide sequence encodes a protein having β-glucosidase activity;
(d) polynucleotides comprising a nucleotide sequence the complementary strand of which hybridizes to the polynucleotide of any one of (a) to (c), wherein said nucleotide sequence encodes a protein having β-glucosidase activity; and
(e) polynucleotides comprising a nucleotide sequence that deviates from the nucleotide sequence defined in (d) by the degeneracy of the genetic code.

Consequently, the present invention relates to polynucleotides encoding a polypeptide having beta-glucosidase activity, said polynucleotides preferably encoding a polypeptide comprising the amino acid sequence indicated in SEQ ID NO: 2. This polypeptide belongs to the protein class of family 1 glycoside hydrolases (Henrissat, Curr. Opin. Struct. Biol. 7 (1997), 637-644).

The present invention is based on the finding that a mutant plant in which the gene encoding the polypeptide of the invention is inactivated due to the presence of a nonsense mutation shows a significant increase of susceptibility to non-host pathogens.

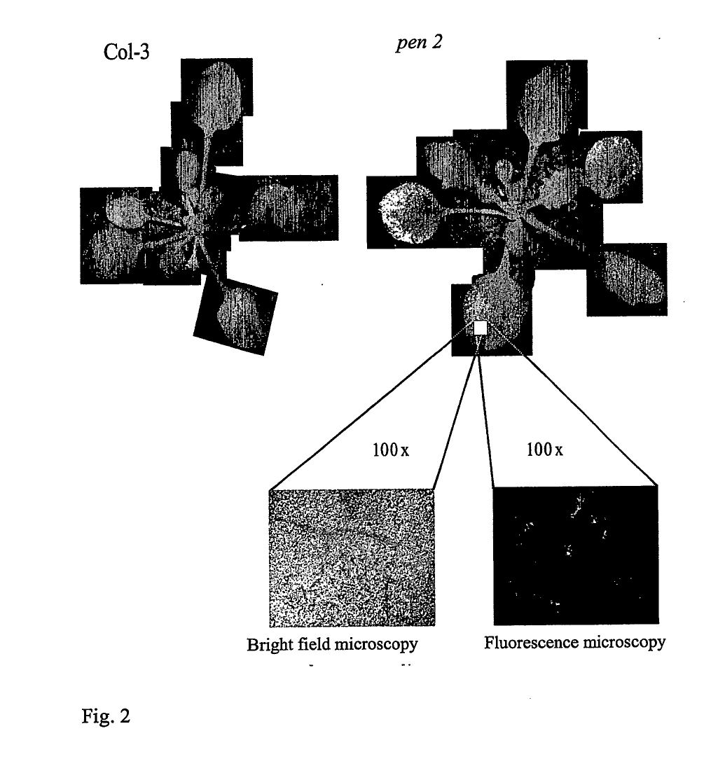

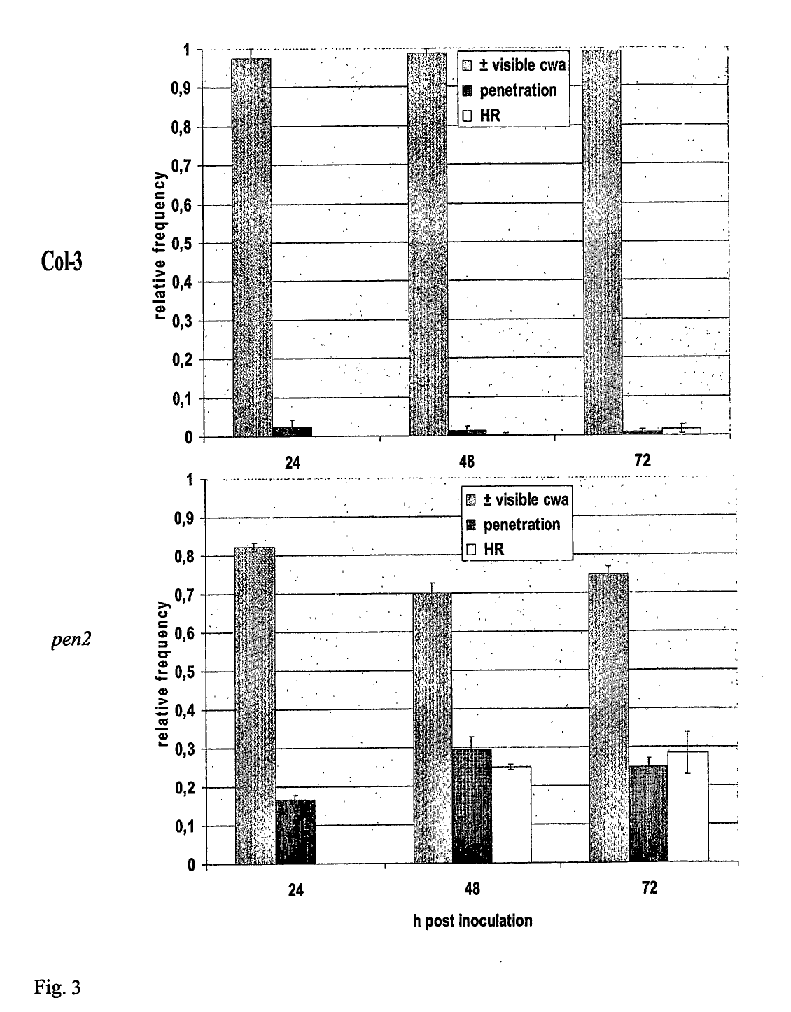

This plant mutant, Arabidopsis pen2, has been produced in connection with the present invention by chemically mutagenizing Arabidopsis Col-3 plants (see Example 2). The M2 generation thereof has been phenotypically screened for an increase of penetration incidences. For this purpose, a screening method has been developed by which M2 plants were inoculated with barley powdery mildew (Bgh) and examined under stereomicroscope and UV light for sites of autofluorescence (FIG. 2). Each site of autofluorescence was interpreted as a site of successful penetration by the pathogen resulting in a hypersensitive reaction that is recognizable by a release of polyphenolic compounds giving rise to autofluorescence. Thus, a significant increase of the amount of autofluorescence sites was indicative for a reduced ability of the plant to prevent pathogen penetration through the cell wall. A closer cytological analysis of the pen2 plants indeed revealed that the frequency of successful pathogen penetrations is about 30% of the incidents of pathogen attack which is a 15-fold increase as compared to a corresponding frequency of about 2% in wild-type plants (see Example 2 and FIG. 3). Interestingly, the capacity to ultimately successfully colonize the host plant remained unaltered in the pen2 mutant as compared to wild-type plants. Thus, the ability to counter pathogen penetration by a hypersensitive response, e.g. leading to programmed cell death, was not affected by the pen2 mutation. Based on these observations, it is concluded that the PEN2 protein is involved in non-host resistance in Arabidopsis. Its function is exerted at an early stage of disease development and its site of action is, at least in part, the cell wall.

Importantly, the defense of pen2 mutant plants is compromised with respect to different fungal pathogens (see Example 3). In response to challenges with Phytophthora infestans and wheat powdery mildew (Bgt), pen2 plants showed strikingly enhanced penetration frequencies and cell death incidents when compared to corresponding wild-type plants. Upon challenges with Pyricularia grisea, to which wild-type plants do not respond in any detectable way, pen2 plants show cell wall appositions at about 10% of the interaction sites. These results show that PEN2 is involved in non-host resistance to a broad scale of pathogens, at least to fungal pathogens of the taxonomic divisions oomycetes (P. infestans) and ascomycetes (Bgh, Bgt and P. grisea). In addition, it is evident that PEN2 has a dual role, namely playing part in static and in inducible defense. In P. grisea, the inactivation of PEN2 leads to the break-down of the static cell wall barrier so that active defense mechanisms become necessary in order to counter pathogen attack. With respect to Bgh, Bgt and P. infestans, additionally the inducible mechanisms are weakened in pen2 mutant plants.

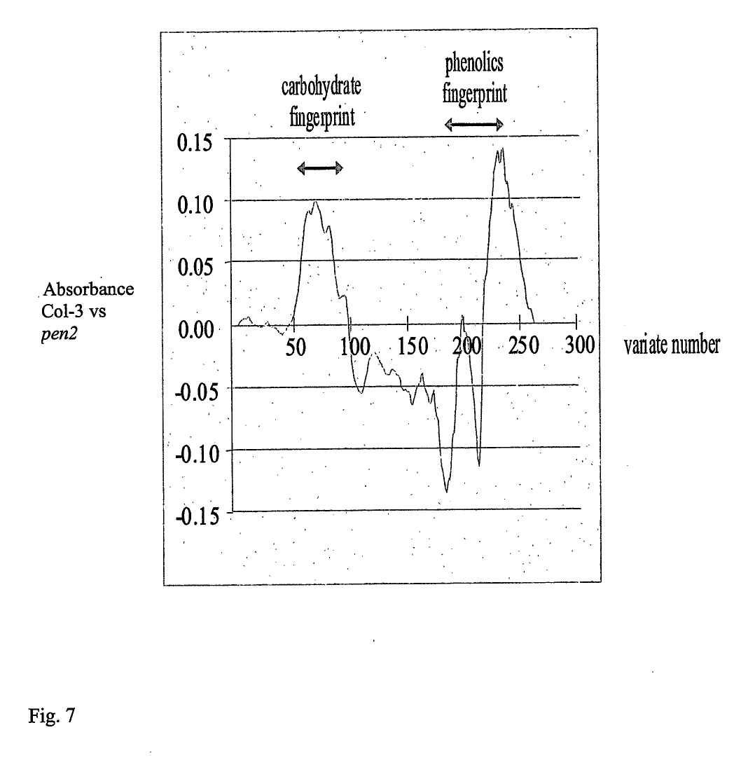

A further clue to the function of PEN2 has been revealed by analyzing the cell wall composition of uninfected wild-type and pen2 plants (see Example 4). The difference FTIR spectrum shown in FIG. 7 indicates significant constitutive changes in the cell wall composition of pen2 plants in the carbohydrate and phenolics fingerprint. This additionally evidences that a site of action of PEN2 is the cell wall.

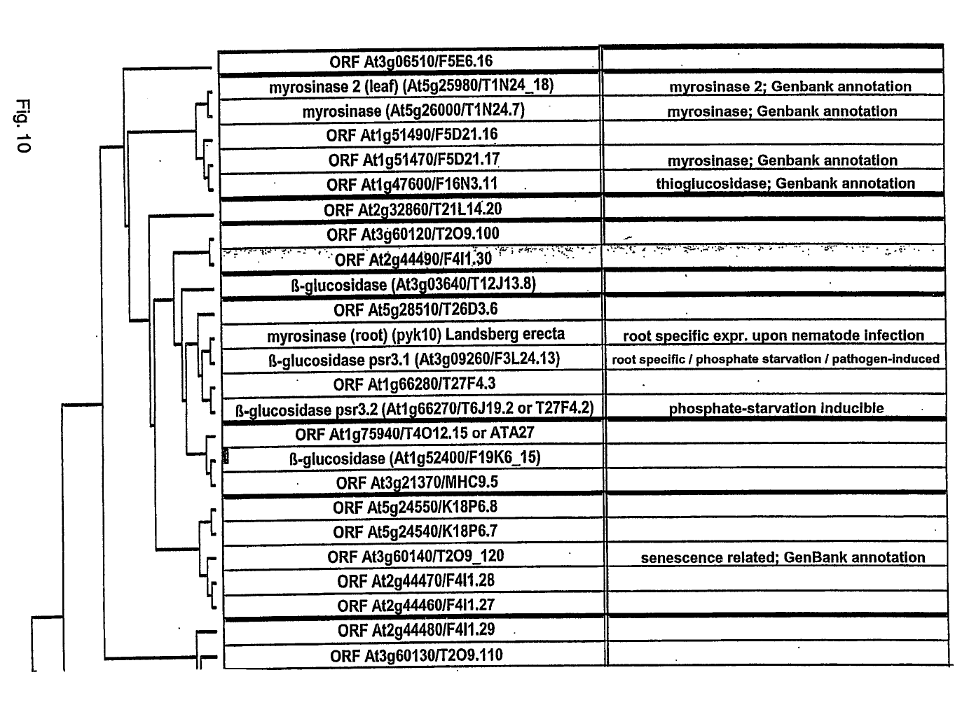

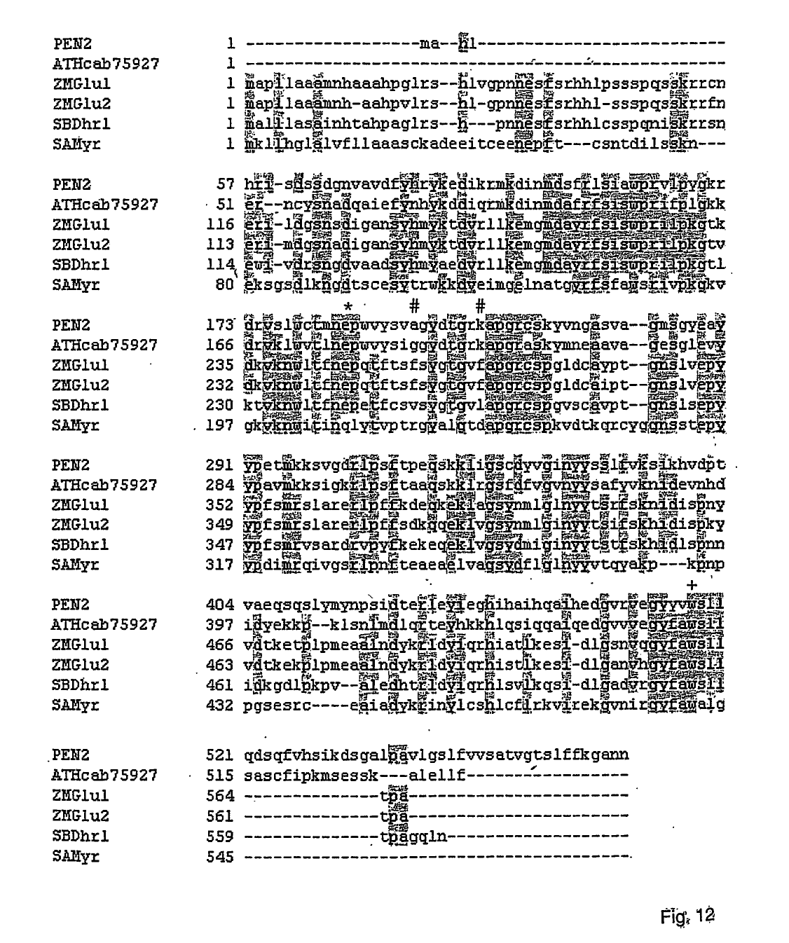

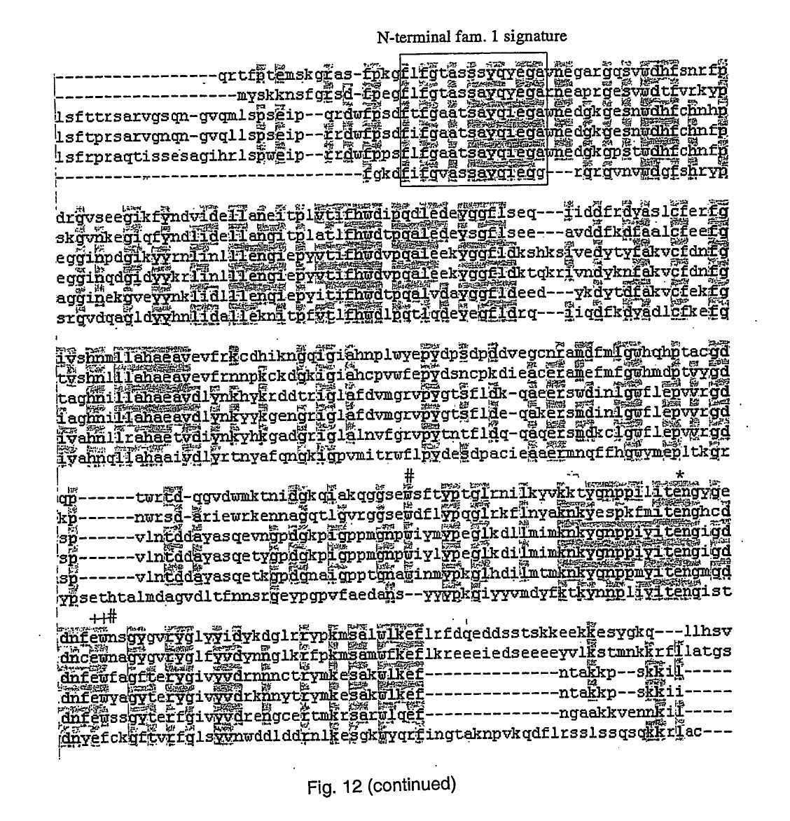

By map-based cloning, the site of the pen2 mutation could be located to an open reading frame designated At2g44490 of chromosome 2 of the Arabidopsis genome (see Example 5). According to its structural characteristics, PEN2 can be assigned to the protein class of family 1 glycoside hydrolases. One such structural characteristic is the presence of the family 1 N-terminal signature (see FIG. 9). Another characteristic is the presence of two glutamate residues at position 183 and 398 of SEQ ID NO:2. They lie within conserved sequence motifs which are described to be typical for family 1 glycoside hydrolases (TFNEP and (I/V)TENG; SEQ ID NOs:3 and 4). In PEN2, the residue F is replaced by M which probably will not affect catalytic activity since both amino acids are apolar. The structural characteristics of PEN2 in comparison to other family 1 glycoside hydrolases are depicted in Table 1 and FIG. 12. In Czjzek (Proc. Natl. Acad. Sci. USA 97 (2000), 13555-13560) structural characteristics of family 1 glycoside hydrolases are summarized and are referred to herein. The glutamate residues E183 and E398 which are catalytically active are conserved throughout the family except for myrosinases which show Q and E. Amino acid residues in PEN2 which are predicted to be involved in determining substrate, preferably aglycone specificity are A190, K197, W370 and N459. This combination of amino acid residues is unique amongst all deduced Arabidopsis family 1 glycosyl hydrolases (Table 1), suggesting that PEN2 has a unique substrate specificity. Predicted residues responsible for glucose binding are: W450, E457 and W458.

TABLE 1
Compilation of predicted catalytically active and aglycone specificity determining residues in all known 47 Arabidopsis
family 1 glycosyl hydrolases including PEN2. cat. = catalytically active residues; agl. sp. = aglycone specificity
determining residues; gluc. Bind. = glucose binding residues (according to Czjzek et al., 2000).

| | | | | | | | | gluc. | gluc. | gluc. | agl. |
|---|---|---|---|---|---|---|---|---|---|---|---|
| Gene | Protein-ID | length | cat. | agl. sp. | agl. sp. | agl. sp. | cat. | bind. | bind. | bind. | spec. |
| ORF At3g06510/F5E6.16 | AF219380 | 618 | E | L | w | — | E | W | E | W | A |
| myrosinase 2 (leaf) | F149413 | 536 | Q | R | d | — | E | W | E | F | C |
| (At5g25980/T1N24_18) | | | | | | | | | | | |
| myrosinase (At5g26000/T1N24.7) | AAC18869 | 541 | Q | R | d | — | E | W | E | F | C |
| ORF At1g51490/F5D21.16 | AC024261_9 | 421 | Q | K | y | — | E | W | E | F | G |
| ORF At1g51470/F5D21.17 | AC024261_15 | 465 | Q | K | y | — | E | W | E | F | G |
| ORF At1g47600/F16N3.11 | C007519 | 510 | Q | K | y | — | E | W | E | F | G |
| ORF At2g32860/T21L14.20 | AAB91979 | 614 | E | A | K | — | E | W | E | W | D |
| ORF At3g60120/T2O9.100 | CAB75927 | 534 | E | G | K | W | E | W | E | W | N |
| PEN2/ORF At2g44490/F4I1.30 | AAC16095 | 560 | E₁₈₃ | A₁₉₀ | K₁₉₇ | W₃₇₀ | E₃₉₈ | W₄₅₀ | E₄₅₇ | W₄₅₈ | N₄₅₉ |
| β-glucosidase (At3g03640/T12J13.8) | AAC31962 | 531 | E | G | K | K | G | S | E | W | E |
| ORF At5g28510/T26D3.6 | AAF88017 | 531 | E | A | K | P | E | W | E | W | Q |
| myrosinase (root) (pyk10) Landsberg | CAA61592 | 524 | E | A | K | A | E | W | E | W | Q |
| erecta | | | | | | | | | | | |
| β-glucosidase psr3.1 | AAB38783 | 524 | E | A | K | A | E | W | E | W | Q |
| (At3g09260/F3L24.13) | | | | | | | | | | | |
| ORF At1g66280/T27F4.3 | AC020665 | 524 | E | A | K | A | E | W | E | W | Q |
| β-glucosidase psr3.2 | AAB64244 | 524 | E | A | K | A | E | W | E | W | Q |
| (At1g66270/T6J19.2 or T27F4.2) | | | | | | | | | | | |
| ORF At1g75940/T4O12.15 or ATA27 | AAC39504 | 535 | E | A | K | K | E | W | E | W | Q |
| β-glucosidase (At1g52400/F19K6_15) | AF183827 | 528 | E | A | K | K | E | W | E | W | Q |
| ORF At3g21370/MHC9.5 | AAL24252 | 530 | E | S | K | K | E | W | E | W | Q |
| ORF At5g24550/K18P6.8 | BAB11207 | 531 | E | S | K | I | E | W | E | W | E |
| ORF At5g24540/K18P6.7 | BAB11206 | 534 | E | S | K | I | E | W | E | W | E |
| ORF At3g60140/T2O9_120 | AAG23719 | 577 | E | A | K | F | E | W | E | W | E |
| ORF At2g44470/F4I1.28 | AAC16093 | 591 | E | A | K | K | E | W | E | W | E |
| ORF At2g44460/F4I1.27 | AAC16092 | 577 | E | A | K | I | E | W | E | W | E |
| ORF At2g44480/F4I1.29 | AAC16094 | 517 | E | L | I | W | E | W | E | W | E |
| ORF At3g60130/T2O9.110 | AAK76627 | 514 | E | E | K | W | E | W | E | W | S |
| ORF At2g44450/F4I1.26 | AAC16091 | 506 | E | Q | M | W | E | W | E | W | A |
| ORF At2g25630 (fragment) | AC006053 | 384 | E | Q | M | — | — | — | — | — | — |
| ORF At5g42260/K5J14.6 | BAB10199 | 507 | E | Q | M | W | E | W | E | W | A |
| ORF At5g44640/K15C23.9 | BAA98117 | 507 | E | Q | M | W | E | W | E | W | A |
| ORF At1g02850/F22D16_15 | C009525 | 520 | E | G | T | W | E | W | E | I | F |
| ORF At3g62740/F26K9_170 | AAK59461 | 491 | E | G | R | — | — | F | D | i | — |
| ORF At3g62750/F26K9_180 | CAB83125 | 440 | E | A | — | W | E | — | — | — | — |
| ORF At4g27830/T27E11.70 | CAB49371 | 517 | E | G | S | W | E | W | E | I | L |
| ORF At4g27820/T27E11.60 | CAB43970 | 498 | E | G | A | W | E | W | E | i | L |
| ORF At5g16580/MTG13.2 (fragment) | BAB10185 | 341 | E | G | S | W | E | — | — | — | — |
| ORF At4g22100/F1N20.200 | CAA18113 | 468 | E | G | T | W | E | W | E | I | V |
| ORF At1g60270/T13D8.16 | AAC24060 | 477 | E | G | S | — | E | W | E | i | K |
| ORF At1g60090/T2K10.15 | AAD14488 | 528 | E | G | T | W | E | W | E | I | L |
| ORF At1g60260/T13D8.15 | AAC24061 | 449 | E | G | T | W | E | W | E | I | I |
| ORF At4g21760/F17L22.220 | CAB36820 | 520 | E | G | Y | — | E | — | — | — | — |
| ORF At1g61820/F8K4.3 | AAC28502 | 527 | E | L | F | W | E | W | E | W | L |
| ORF At1g61810/F8K4.2 | AAC28501 | 520 | E | L | F | W | E | W | E | W | L |
| ORF At5g36890/MLF18.1 | BAB11630 | 475 | E | N | F | W | E | W | E | W | A |
| ORF At5g54570/MRB17.7 | BAB09336 | 520 | E | Q | Q | W | E | W | E | W | N |
| ORF At1g26560/T1K7.7 | AC013427 | 510 | E | Q | Q | W | E | W | E | W | A |
| ORF At3g18070/MRC8.5 | BAB02019 | 502 | E | L | F | W | E | W | E | W | L |
| ORF At3g18080/MRC8.6 | AF360240 | 512 | E | L | F | W | E | W | E | W | L |

The classification of PEN2 as a beta-glucosidase is based on its overall homology with the closest relatives in the protein family as well as on the presence of the N-terminal signature (FIG. 9). In the GenBank/EMBL database entry AC004521, the pen2 ORF has already been identified as a putative beta-glucosidase. However, it was not yet clear whether this ORF is actually expressed, let alone that nothing was known on a crucial role of the PEN2 protein encoded by this ORF in non-host resistance.

From the results discussed above, it is evident that the PEN2 protein is involved in the establishment of non-host resistance in plants. Thus, it can be expected that an increase of the activity of this protein in plants may establish or enhance resistance, preferably non-host resistance in said plants.

In the context of the present invention, the term “pathogen” refers to organisms that attack plants. It includes, for example, bacteria, viruses, viroids, fungi and protozoa. Among fungal pathogens, species belonging to the taxonomic groups oomycota, ascomycetes and basidiomycetes (see for reference, e.g., Strasburger, Lehrbuch der Botanik, 33^{rd} edition, 1991, G. Fischer Verlag, Stuttgart, Jena, New-York) are pathogens of particular interest in the context of the present invention. Examples of important pathogens are Phytophthora infestans (the causal agent of potato late blight disease), Phytophthora sojae (root rot in soybean), Peronospora parasitica (downy mildew), Magnaporthe grisea (rice blast disease), Erysiphe spp (powdery mildew), Pseudomonas syringae (bacterial blight), Erwinia amylovora (fire blight disease), Erwinia carotovora (soft rot), Botrytis cinerea (downy mildew of grape), Rhizoctonia solani and Pythium debaryanum (agents of seedling blight or damping off disease).

The term “susceptibility” refers to the capacity of a given pathogen to grow on or in the tissue of a plant. In particular, “susceptibility” refers to the growth of a pathogen on the epidermal surface and from there into the epidermis and subepidermal tissue, e.g. the mesophyll. Thus, the term “susceptibility” also covers incidents of pathogen attacks where the pathogen grows for a certain while on the host plant, however, without being capable to take up nutrients from the host and therefore without successfully colonising the host plant. In particular, successful colonization is characterized by completing that part of the pathogen's life cycle which takes place on the plant host. With regard to fungal pathogens, like for instance powdery mildew, such a successful colonisation is for instance apparent from the formation of a haustorium and of secondary hyphae.

The term “non-host” refers to plant-pathogen interactions between all individuals of one plant species and all individuals of one pathogen species, wherein none of such interactions lead to a successful colonisation of the pathogen on the plant. Thus, the term “non-host pathogen” refers to a pathogen species to which a certain plant species shows non-host resistance, i.e. all individuals of the plant species are resistant against all individuals of the pathogen species.

In connection with the present invention, the term “resistant” or “resistance” refers to the property of a given plant or plant species to protect itself against an attack by a certain pathogen, whereby said protection may range from a delay to a complete inhibition of disease development. Preferably, “resistance” refers to an effective block of pathogen growth on or in said plant or plant species so that the pathogen is not able to successfully colonize the plant or plant species. Generally, resistance involves an interplay of various means that aim at blocking penetration of the pathogen into the plant. This may refer to static properties of the plant, i.e. structural, chemical or other characteristics of the plant that prevent or reduce pathogen penetration and which are constitutively present in the plant, i.e. independent of whether there is a pathogen attack or not. On the other hand, such means may also be inducible mechanisms with which a plant reacts to pathogen attack. Among these mechanisms, cell wall appositions, the expression of pathogen-related proteins and hypersensitive cell death are prominent examples. In connection with the present invention, resistance is preferably exerted at the level of cell wall penetration. This means that the provisions of the invention preferably improve or establish resistance against a pathogen by decreasing its capacity to overcome a cell wall barrier, which preferably is the outer cell wall of the epidermis or rhizodermis. It is furthermore preferred that the provisions of the invention improve or establish resistance against a pathogen by improving static defense (i.e. the capacity of a plant to prevent cell wall penetration by a pathogen without detectably inducing an inducible defense mechanism) and/or by improving one or more inducible defense mechanisms, in particular cell wall remodelling. The term “cell wall remodelling” refers to any structural changes of a cell wall that may take place in a plant as a response to pathogen attack such as cell wall apposition or encasement of parts of the pathogen such as a haustorium in material comprising callose.

Furthermore, the term “improved resistance” refers to a significant reduction of susceptibility to a pathogen in plants treated according to the provisions of the present invention as compared to corresponding untreated plants. In particular, such a reduction of susceptibility may be evident from a significant reduction of penetration events and/or a significant reduction of hypersensitive reactions as for instance visible by fluorescence detection. Preferably, such a reduction of susceptibility of a plant so-treated is by at least 10%, more preferably at least 20%, still more preferably by at least 50%, even more preferably by at least 80% and most preferably to approximately 100% as compared to an untreated plant and with respect to the number of penetration events and/or hypersensitive reactions. The term “treated according to the provisions of the invention” refers to any means of enhancing the activity of the protein of the invention in the respective plant such as by overexpressing the protein in transgenic plants or by adding exogenous substrate. Said term may also refer to the administration of the enzymatic product of the protein of the invention which may give rise to an effect similar to directly enhancing the activity of the protein of the invention.

The invention in particular relates to polynucleotides containing the nucleotide sequence indicated under SEQ ID NO: 1 or encoding the amino acid sequence shown under SEQ ID NO: 2 or a part thereof having beta-glucosidase activity. The term “beta-glucosidase activity” refers to the activities of the polypeptide with the amino acid sequence shown in SEQ ID NO:2. In particular, this term refers to the enzymatic activity acting on substrates containing at least one glycosidic bond such as for instance oligosaccharides, polysaccharides or conjugates between at least one glycosidic group (also called “glycone” or “carbohydrate”) and a non-glycosidic moiety (also called “aglycone” or “non-carbohydrate”), whereby this bond is hydrolyzed by the hydrolytic activity of the polypeptide. The polypeptide of the invention belongs to the class of family 1 glycoside hydrolases (http://afmb.cnrs-mrs.fr/˜cazy/CASY and Henrissat, Curr. Opin. Struct. Biol. 7 (1997), 637-644). Enzymes of this class either hydrolyze O-linked beta-glycosidic bonds (beta-D-glucoside glycohydrolase; EC 3.2.1.21) or S-linked beta-glycosidic bonds (myrosinase or beta-D-thioglucoside glycohydrolase; EC 3.2.3.1). The structural characteristics of the polypeptide of the invention are described above in connection with PEN2 and are depicted in Table 1. In addition, the activity of the polypeptide in plants also manifests itself in the cell wall composition. Thus, it may for instance also be determined whether the protein of the invention is expressed and active in a given plant tissue sample by comparing the cell wall composition of said sample with the cell wall composition from plant tissue where the protein of the invention is known to be active and/or with the cell wall composition from plant tissue where the protein of the invention is known not to be active. Corresponding techniques for analyzing the composition of cell walls are known by a person skilled in the art and include for instance Fourier transform infrared (FT-IR) microspectroscopy. By this technique, it is possible to detect significant differences of the cell wall composition, in particular with regard to carbohydrates and phenolics (see also Example 4).

Moreover, the present invention relates to polynucleotides which encode a polypeptide having beta-glucosidase activity and the complementary strand of which hybridizes with a polynucleotide mentioned in sections (a) to (c), above.

The present invention also relates to polynucleotides which encode a polypeptide, which has a homology, that is to say a sequence identity, of at least 30%, preferably of at least 40%, more preferably of at least 50%, even more preferably of at least 60% and particularly preferred of at least 70%, especially preferred of at least 80% and even more preferred of at least 90% to the entire amino acid sequence as indicated in SEQ ID NO: 2, the polypeptide having beta-glucosidase activity.

Moreover, the present invention relates to polynucleotides which encode a polypeptide having beta-glucosidase activity and the nucleotide sequence of which has a homology, that is to say a sequence identity, of at least 40%, preferably of at least 50%, more preferably of at least 60%, even more preferably of more than 65%, in particular of at least 70%, especially preferred of at least 80%, in particular of at least 90% and even more preferred of at least 95% when compared to the coding region of the sequence shown in SEQ ID NO: 1.

It is particularly preferred that polynucleotides of the invention that encode a polypeptide having beta-glucosidase show the structural characteristics described above for PEN2 and depicted in Table 1.

The present invention also relates to polynucleotides, which encode a polypeptide having beta-glucosidase activity and the sequence of which deviates from the nucleotide sequences of the above-described polynucleotides due to the degeneracy of the genetic code.

The invention also relates to polynucleotides comprising a nucleotide sequence which is complementary to the whole or a part of one of the above-mentioned sequences.

In the context of the present invention the term “hybridization” means hybridization under conventional hybridization conditions, preferably under stringent conditions, as for instance described in Sambrook and Russell (2001), Molecular Cloning: A Laboratory Manual, CSH Press, Cold Spring Harbor, N.Y., USA. In an especially preferred embodiment, the term “hybridization” means that hybridization occurs under the following conditions:
Hybridization buffer: 2×SSC; 10× Denhardt solution (Fikoll 400+PEG+BSA; ratio 1:1:1); 0.1% SDS; 5 mM EDTA; 50 mM Na₂HPO₄;
250 μg/ml of herring sperm DNA; 50 μg/ml of tRNA; or
0.25 M of sodium phosphate buffer, pH 7.2;
1 mM EDTA
7% SDS
Hybridization temperature T=60° C.
Washing buffer: 2×SSC; 0.1% SDS
Washing temperature T=60° C.

Polynucleotides which hybridize with the polynucleotides of the invention can, in principle, encode a polypeptide having beta-glucosidase activity from any organism expressing such polypeptides or can encode modified versions thereof.

Polynucleotides which hybridize with the polynucleotides disclosed in connection with the invention can for instance be isolated from genomic libraries or cDNA libraries of bacteria, fungi, plants or animals. Preferably, such polynucleotides are from plant origin, particularly preferred from a plant belonging to the dicotyledons, more preferably from the family of Brassicaceae. Preferably, the polynucleotide of the invention is not a polynucleotide with or comprising the nucleotide sequence shown in SEQ ID NO:1, and is a variant of such a polynucleotide as described herein. Preferably, the polynucleotide of the invention is a variant, preferably an ortholog of a polynucleotide comprising SEQ ID NO:1 and may for example comprise a nucleotide sequence that originates from an agronomically important crop species such as from rape seed or cabbage varieties. Alternatively, such polynucleotides can be prepared by genetic engineering or chemical synthesis.

Such hybridizing polynucleotides may be identified and isolated by using the polynucleotides described hereinabove or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Polynucleotides comprising the same or substantially the same nucleotide sequence as indicated in SEQ ID NO: 1 or parts thereof can, for instance, be used as hybridization probes. The fragments used as hybridization probes can also be synthetic fragments which are prepared by usual synthesis techniques, and the sequence of which is substantially identical with that of a polynucleotide according to the invention.

The molecules hybridizing with the polynucleotides of the invention also comprise fragments, derivatives and allelic variants of the above-described polynucleotides encoding a polypeptide having beta-glucosidase activity. Herein, fragments are understood to mean parts of the polynucleotides which are long enough to encode the described polypeptide, preferably showing the biological activity of a polypeptide of the invention as described above. In this context, the term derivative means that the sequences of these molecules differ from the sequences of the above-described polynucleotides in one or more positions and show a high degree of homology to these sequences, preferably within the preferred ranges of homology mentioned above.

Preferably, the degree of homology is determined by comparing the respective sequence with the nucleotide sequence of the coding region of SEQ ID NO: 1. When the sequences which are compared do not have the same length, the degree of homology preferably refers to the percentage of nucleotide residues in the shorter sequence which are identical to nucleotide residues in the longer sequence. The degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 0AS UK (support@dnastar.com) and is accessible at the server of the EMBL outstation.

When using the Clustal analysis method to determine whether a particular sequence is, for instance, 80% identical to a reference sequence the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Preferably, the degree of homology of the hybridizing polynucleotide is calculated over the complete length of its coding sequence. It is furthermore preferred that such a hybridizing polynucleotide, and in particular the coding sequence comprised therein, has a length of at least 300 nucleotides, preferably at least 500 nucleotides, more preferably of at least 750 nucleotides, even more preferably of at least 1000 nucleotides, particularly preferred of at least 1500 nucleotides and most preferably of at least 2000 nucleotides.

Preferably, sequences hybridizing to a polynucleotide according to the invention comprise a region of homology of at least 90%, preferably of at least 93%, more preferably of at least 95%, still more preferably of at least 98% and particularly preferred of at least 99% identity to an above-described polynucleotide, wherein this region of homology has a length of at least 500 nucleotides, more preferably of at least 750 nucleotides, even more preferably of at least 1000 nucleotides, particularly preferred of at least 1500 nucleotides and most preferably of at least 2000 nucleotides.

Homology, moreover, means that there is a functional and/or structural equivalence between the corresponding polynucleotides or polypeptides encoded thereby. Polynucleotides which are homologous to the above-described molecules and represent derivatives of these molecules are normally variations of these molecules which represent modifications having the same biological function. They may be either naturally occurring variations, for instance sequences from other ecotypes, varieties, species, etc., or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, insertion and/or recombination.

The polypeptides encoded by the different variants of the polynucleotides of the invention possess certain characteristics they have in common. These include for instance biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

The biological activity of a polypeptide of the invention, in particular the capacity to hydrolyze the glycosidic bond in a substrate for which it is specific can be tested in conventional enzyme assays using the substrate of the polypeptide or a suitable modified form thereof.

The invention also relates to oligonucleotides specifically hybridizing to a polynucleotide of the invention. Such oligonucleotides have a length of preferably at least 10, in particular at least 15, and particularly preferably of at least 50 nucleotides. Advantageously, their length does not exceed a length of 1000, preferably 500, more preferably 200, still more preferably 100 and most preferably 50 nucleotides. They are characterized in that they specifically hybridize to the polynucleotides of the invention, that is to say that they do not or only to a very minor extent hybridize to nucleic acid sequences encoding another beta-glucosidase. The oligonucleotides of the invention can be used for instance as primers for amplification techniques such as the PCR reaction or as a hybridization probe to isolate related genes. The hybridization conditions and homology values described above in connection with the polynucleotide encoding a polypeptide having beta-glucosidase activity may likewise apply in connection with the oligonucleotides mentioned herein.

The polynucleotides of the invention can be DNA molecules, in particular genomic DNA or cDNA. Moreover, the polynucleotides of the invention may be RNA molecules. The polynucleotides of the invention can be obtained for instance from natural sources or may be produced synthetically or by recombinant techniques, such as PCR.

In another aspect, the present invention relates to recombinant nucleic acid molecules comprising the polynucleotide of the invention described above. The term “recombinant nucleic acid molecule” refers to a nucleic acid molecule which contains in addition to a polynucleotide of the invention as described above at least one further heterologous coding or non-coding nucleotide sequence. The term “heterologous” means that said polynucleotide originates from a different species or from the same species, however, from another location in the genome than said added nucleotide sequence. The term “recombinant” implies that nucleotide sequences are combined into one nucleic acid molecule by the aid of human intervention. The recombinant nucleic acid molecule of the invention can be used alone or as part of a vector.

For instance, the recombinant nucleic acid molecule may encode the polypeptide having beta-glucosidase activity fused to a marker sequence, such as a peptide, which facilitates purification of the fused polypeptide. The marker sequence may for example be a hexa-histidine peptide, such as the tag provided in a pQE vector (Qiagen, Inc.) which provides for convenient purification of the fusion polypeptide. Another suitable marker sequence may be the HA tag which corresponds to an epitope derived from influenza hemagglutinin polypeptide (Wilson, Cell 37 (1984), 767). As a further example, the marker sequence may be glutathione-S-transferase (GST) which, apart from providing a purification tag, enhances polypeptide stability, for instance, in bacterial expression systems.

In a preferred embodiment, the recombinant nucleic acid molecules further comprise expression control sequences operably linked to the polynucleotide comprised by the recombinant nucleic acid molecule, more preferably these recombinant nucleic acid molecules are expression cassettes. The term “operatively linked”, as used throughout the present description, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in prokaryotic as well as in eukaryotic cells, preferably in plant cells, are well known to those skilled in the art. They encompass promoters, enhancers, termination signals, targeting signals and the like. Examples are given further below in connection with explanations concerning vectors. In the case of eukaryotic cells, expression control sequences may comprise poly-A signals ensuring termination of transcription and stabilization of the transcript, for example, those of the 35S RNA from Cauliflower Mosaic Virus (CaMV) or the nopaline synthase gene from Agrobacterium tumefaciens. Additional regulatory elements may include transcriptional as well as translational enhancers. A plant translational enhancer often used is the CaMV omega sequences. Similarly, the inclusion of an intron (e.g. intron-1 from the shrunken gene of maize) has been shown to increase expression levels by up to 100-fold (Mait, Transgenic Research 6 (1997), 143-156; Ni, Plant Journal 7 (1995), 661-676).

Moreover, the invention relates to vectors, in particular plasmids, cosmids, viruses, bacteriophages and other vectors commonly used in genetic engineering, which contain the above-described polynucleotides of the invention. In a preferred embodiment of the invention, the vectors of the invention are suitable for the transformation of fungal cells, cells of microorganisms such as yeast or bacterial cells, animal cells or, in particular, plant cells. In a particularly preferred embodiment such vectors are suitable for stable transformation of plants.

In another preferred embodiment, the vectors further comprise expression control sequences operably linked to said polynucleotides contained in the vectors. These expression control sequence may be suited to ensure transcription and synthesis of a translatable RNA in prokaryotic or eukaryotic cells.

The expression of the polynucleotides of the invention in prokaryotic or eukaryotic cells, for instance in Escherichia coli, is interesting because it permits a more precise characterization of the biological activities of the encoded polypeptide. In particular, recombinantly expressed polypeptide may be used to identify substrate compounds that are hydrolyzed by its activity. Moreover, it is possible to express these polypeptides in such prokaryotic or eukaryotic cells which are free from interfering polypeptides. In addition, it is possible to insert different mutations into the polynucleotides by methods usual in molecular biology (see for instance Sambrook and Russell (2001), Molecular Cloning: A Laboratory Manual, CSH Press, Cold Spring Harbor, N.Y., USA), leading to the synthesis of polypeptides possibly having modified biological properties. In this regard it is on the one hand possible to produce deletion mutants in which polynucleotides are produced by progressive deletions from the 5′ or 3′ end of the coding DNA sequence, and said polynucleotides lead to the synthesis of correspondingly shortened polypeptides.

On the other hand, the introduction of point mutations is also conceivable at positions at which a modification of the amino acid sequence for instance influences the biological activity or the regulation of the polypeptide.

Moreover, mutants possessing a modified substrate or product specificity can be prepared. Furthermore, it is possible to prepare mutants having a modified activity-temperature-profile. Preferably, such mutants show an increased activity. Alternatively, mutants can be prepared the catalytic activity of which is abolished without loosing substrate binding activity. Such a mutant may for example be produced by substituting the amino acid residue corresponding to the glutamate residue at position 183 of SEQ ID NO: 2 by another amino acid residue, e.g. an aspartate residue. This mutagenesis may be carried out as for instance described in Czjzek (Proc. Natl. Sci. USA 97 (2000), 13555-13560). Such inactivated but still binding mutants may be useful for screening for substrate compounds by the so-called substrate trap technique.

Furthermore, in the case of expression in plants, plant tissue or plant cells, the introduction of mutations into the polynucleotides of the invention allows the gene expression rate and/or the activity of the polypeptides encoded by the polynucleotides of the invention to be reduced or increased.

For genetic engineering in prokaryotic cells, the polynucleotides of the invention or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Additionally, the present invention relates to a method for producing genetically engineered host cells comprising introducing the above-described polynucleotides, recombinant nucleic acid molecules or vectors of the invention into a host cell.

Another embodiment of the invention relates to host cells, in particular prokaryotic or eukaryotic cells, genetically engineered with the above-described polynucleotides, recombinant nucleic acid molecules or vectors of the invention or obtainable by the above-mentioned method for producing genetically engineered host cells, and to cells derived from such transformed cells and containing a polynucleotide, recombinant nucleic acid molecule or vector of the invention. In a preferred embodiment the host cell is genetically modified in such a way that it contains a polynucleotide stably integrated into the genome. Preferentially, the host cell of the invention is a bacterial, yeast, fungus, plant or animal cell.

More preferably the polynucleotide can be expressed so as to lead to the production of a polypeptide having beta-glucosidase activity. An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc. The polypeptide according to the present invention can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Polypeptide refolding steps can be used, as necessary, in completing configuration of the polypeptide. Finally, high performance liquid chromatography (HPLC) can be employed for final purification steps.

Accordingly, the present invention also relates to a method for the production of a polypeptide encoded by a polynucleotide of the invention as described above in which the above-mentioned host cell is cultivated under conditions allowing for the expression of the polypeptide and in which the polypeptide is isolated from the cells and/or the culture medium.

Moreover, the invention relates to a polypeptide which is encoded by a polynucleotide according to the invention or obtainable by the above-mentioned method for the production of a polypeptide encoded by a polynucleotide of the invention.

The polypeptide of the present invention may, e.g., be a naturally purified product or a product of chemical synthetic procedures or produced by recombinant techniques from a prokaryotic or eukaroytic host (for example, by bacterial, yeast, higher plant, insect and mammalian cells in culture). Depending upon the host employed in a recombinant production procedure, the polypeptide of the present invention may be glycosylated or may be non-glycosylated. The polypeptide of the invention may also include an initial methionine amino acid residue. The polypeptide according to the invention may be further modified to contain additional chemical moieties not normally part of the polypeptide. Those derivatized moieties may, e.g., improve the stability, solubility, the biological half life or absorption of the polypeptide. The moieties may also reduce or eliminate any undesirable side effects of the polypeptide and the like. An overview for these moieties can be found, e.g., in Remington's Pharmaceutical Sciences (18^{th} ed., Mack Publishing Co., Easton, Pa. (1990)). Polyethylene glycol (PEG) is an example for such a chemical moiety which has been used for the preparation of therapeutic polypeptides. The attachment of PEG to polypeptides has been shown to protect them against proteolysis (Sada et al., J. Fermentation Bioengineering 71 (1991), 137-139). Various methods are available for the attachment of certain PEG moieties to polypeptides (for review see: Abuchowski et al., in “Enzymes as Drugs”; Holcerberg and Roberts, eds. (1981), 367-383). Generally, PEG molecules are connected to the polypeptide via a reactive group found on the polypeptide. Amino groups, e.g. on lysines or the amino terminus of the polypeptide are convenient for this attachment among others.

Furthermore, the present invention also relates to an antibody specifically recognizing a polypeptide according to the invention. The antibody can be monoclonal or polyclonal and can be prepared according to methods well known in the art. The term “antibody” also comprises fragments of an antibody which still retain the binding specificity.

The polypeptide according to the invention, its fragments or other derivatives thereof, or cells expressing them can be used as an immunogen to produce antibodies thereto. The present invention in particular also includes chimeric, single chain, and humanized antibodies, as well as Fab fragments, or the product of an Fab expression library. Various procedures known in the art may be used for the production of such antibodies and fragments.

Antibodies directed against a polypeptide according to the present invention can be obtained, e.g., by direct injection of the polypeptide into an animal or by administering the polypeptide to an animal, preferably a non-human animal. The antibody so obtained will then bind the polypeptide itself. In this manner, even a sequence encoding only a fragment of the polypeptide can be used to generate antibodies binding the whole native polypeptide. Such antibodies can then, e.g., be used to isolate the polypeptide from tissue expressing that polypeptide or to detect it in a probe. For the preparation of monoclonal antibodies, any technique which provides antibodies produced by continuous cell line cultures can be used. Examples for such techniques include the hybridoma technique (Köhler and Milstein, Nature 256 (1975), 495-497), the trioma technique, the human B-cell hybridoma technique (Kozbor et al., Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to immunogenic polypeptides according to the present invention. Furthermore, transgenic mice may be used to express humanized antibodies directed against immunogenic polypeptides of the present invention.

Furthermore, the invention relates to a method for producing a transgenic plant comprising the steps of
(a) introducing at least one of the above-described polynucleotides, recombinant nucleic acid molecules or vectors of the invention into the genome of a plant cell; and
(b) regenerating the cell of (a) to a transgenic plant.
   Optionally, the method may further comprise step (c) producing progeny from the plants produced in step (b).

In a further aspect, the invention relates to transgenic plants or plant tissue comprising plant cells which are genetically engineered with the polynucleotide of the invention or which contain the recombinant nucleic acid molecule or the vector of the invention or to transgenic plants obtainable by the method mentioned above.

Preferably, in the transgenic plant of the invention, the polynucleotide of the invention is expressed at least in one part, i.e. organ, tissue or cell type, of the plant. Preferably, this expression leads to an increase of beta-glucosidase activity in the cells which express said polynucleotide or in the environment of such cells, e.g. in the apoplast, in particular in the cell wall. Increase of activity can be detected for instance by measuring the amount of transcript and/or protein in the transformed cell, tissue or plant in comparison to corresponding measurements at non-transformed plant cells, tissue or plants. According to the teachings of the present invention, an increase of the activity of the polypeptide of the invention in transgenic plants leads to an increase of resistance against a plant pathogen to which a corresponding wild-type plant is susceptible or at least more susceptible.

The polynucleotide introduced into the transgenic plant can in principle be expressed in all or substantially all cells of the plant. However, it is also possible that it is only expressed in certain parts, organs, cell types, tissues etc. Moreover, it is possible that expression of the polynucleotide only takes place upon induction, at a certain developmental stage or, as it may be preferred in some embodiments, upon pathogen attack. In a preferred embodiment, the polynucleotide is expressed in those parts of the plant that are exposed to pathogen attack, for example the epidermis or the rhizodermis.

In order to be expressed, the polynucleotide that is introduced into a plant cell is preferably operatively linked to one or more expression control sequences, e.g. a promoter, active in this plant cell.

The promoter may be homologous or heterologous with regard to its origin and/or with regard to the gene to be expressed. Suitable promoters are for instance the promoter of the 35S RNA of the Cauliflower Mosaic Virus (see for instance U.S. Pat. No. 5,352,605) and the ubiquitin-promoter (see for instance U.S. Pat. No. 5,614,399) which lend themselves to constitutive expression, the patatin gene promoter B33 (Rocha-Sosa et al., EMBO J. 8 (1989), 23-29) which lends itself to a tuber-specific expression in potatoes or a promoter ensuring expression in photosynthetically active tissues only, for instance the ST-LS1 promoter (Stockhaus et al., Proc. Natl. Acad. Sci. USA 84 (1987), 7943-7947; Stockhaus et al., EMBO, J. 8 (1989) 2445-2451), the Ca/b-promoter (see for instance U.S. Pat. No. 5,656,496, U.S. Pat. No. 5,639,952, Bansal et al., Proc. Natl. Acad. Sci. USA 89 (1992), 3654-3658) and the Rubisco SSU promoter (see for instance U.S. Pat. No. 5,034,322; U.S. Pat. No. 4,962,028) or the glutelin promoter from wheat which lends itself to endosperm-specific expression (HMW promoter) (Anderson, Theoretical and Applied Genetics 96, (1998), 568-576, Thomas, Plant Cell 2 (12), (1990), 1171-1180), the glutelin promoter from rice (Takaiwa, Plant Mol. Biol. 30(6) (1996), 1207-1221, Yoshihara, FEBS Lett. 383 (1996), 213-218, Yoshihara, Plant and Cell Physiology 37 (1996), 107-111), the shrunken promoter from maize (Maas, EMBO J. 8 (11) (1990), 3447-3452, Werr, Mol. Gen. Genet. 202(3) (1986), 471-475, Werr, Mol. Gen. Genet. 212(2), (1988), 342-350), the USP promoter, the phaseolin promoter (Sengupta-Gopalan, Proc. Natl. Acad. Sci. USA 82 (1985), 3320-3324, Bustos, Plant Cell 1 (9) (1989), 839-853) or promoters of zein genes from maize (Pedersen et al., Cell 29 (1982), 1015-1026; Quatroccio et al., Plant Mol. Biol. 15 (1990), 81-93). However, promoters which are only activated at a point in time determined by external influences can also be used (see for instance WO 93/07279). In this connection, promoters of heat shock proteins which permit simple induction may be of particular interest. Likewise, artificial and/or chemically inducible promoters may be used in this context. Moreover, seed-specific promoters such as the USP promoter from Vicia faba which ensures a seed-specific expression in Vicia faba and other plants may be used (Fiedler et al., Plant Mol. Biol. 22 (1993), 669-679; Bäumlein et al., Mol. Gen. Genet. 225 (1991), 459-467). Moreover, fruit-specific promoters, such as described in WO 91/01373 may be used too. In one embodiment, promoters which ensure constitutive expression are preferred. However, in another preferred embodiment, the polynucleotide may be operatively linked to a promoter which is inducible upon pathogen attack.

Moreover, the polynucleotide may be linked to a termination sequence which serves to terminate transcription correctly and to add a poly-A-tail to the transcript which is believed to have a function in the stabilization of the transcripts. Such elements are described in the literature (see for instance Gielen et al., EMBO J. 8 (1989), 23-29) and can be replaced at will.

Furthermore, if needed, polypeptide expression can in principle be targeted to any sub-localization of plant cells (e.g. cytosol, plastids, vacuole, mitochondria) or the plant (e.g. apoplast). In order to achieve the localization in a particular compartment, the coding region to be expressed may be linked to DNA sequences encoding a signal sequence (also called “transit peptide”) ensuring localization in the respective compartment. It is evident that these DNA sequences are to be arranged in the same reading frame as the coding region to be expressed. Preferably, signal sequences directing expression into the apoplast are used in connection with the present invention.

In order to ensure the location in the plastids it is conceivable to use one of the following transit peptides: of the plastidic Ferredoxin: NADP+ oxidoreductase (FNR) of spinach which is enclosed in Jansen et al. (Current Genetics 13 (1988), 517-522). In particular, the sequence ranging from nucleotides −171 to 165 of the cDNA sequence disclosed therein can be used which comprises the 5′ non-translated region as well as the sequence encoding the transit peptide. Another example is the transit peptide of the waxy protein of maize including the first 34 amino acid residues of the mature waxy protein (Klösgen et al., Mol. Gen. Genet. 217 (1989), 155-161). It is also possible to use this transit peptide without the first 34 amino acids of the mature protein. Furthermore, the signal peptides of the ribulose bisphosphate carboxylase small subunit (Wolter et al., Proc. Natl. Acad. Sci. USA 85 (1988), 846-850; Nawrath et al., Proc. Natl. Acad. Sci. USA 91 (1994), 12760-12764), of the NADP malat dehydrogenase (Gallardo et al., Planta 197 (1995), 324-332), of the glutathione reductase (Creissen et al., Plant J. 8 (1995), 167-175) or of the R1 protein (Lorberth et al. Nature Biotechnology 16, (1998), 473-477) can be used. In order to ensure the location in the vacuole, it is conceivable to use one of the following transit peptides: the N-terminal sequence (146 amino acids) of the patatin protein (Sonnewald et al., Plant J. 1 (1991), 95-106) or the signal sequences described by Matsuoka and Neuhaus (Journal of Experimental Botany 50 (1999), 165-174); Chrispeels and Raikhel (Cell 68 (1992), 613-616); Matsuoka and Nakamura (Proc. Natl. Acad. Sci. USA 88 (1991), 834-838); Bednarek and Raikhel (Plant Cell 3 (1991), 1195-1206); and Nakamura and Matsuoka (Plant Phys. 101 (1993), 1-5).

In order to ensure the localization in the mitochondria, it is for example conceivable to use the transit peptide described by Braun (EMBO J. 11, (1992), 3219-3227).

In order to ensure the localization in the apoplast, it is conceivable to use one of the following transit peptides: signal sequence of the proteinase inhibitor II-gene (Keil et al., Nucleic Acid Res. 14 (1986), 5641-5650; von Schaewen et al., EMBO J. 9 (1990), 30-33), of the levansucrase gene from Erwinia amylovora (Geier and Geider, Phys. Mol. Plant Pathol. 42 (1993), 387-404), of a fragment of the patatin gene B33 from Solanum tuberosum, which encodes the first 33 amino acids (Rosahl et al., Mol Gen. Genet. 203 (1986), 214-220) or of the one described by Oshima et al. (Nucleic Acid Res. 18 (1990), 181).

The transgenic plants of the invention may, in principle, be plants of any plant species. They may be both monocotyledonous and dicotyledonous plants. Preferably, the plants are useful plants, i.e. commercially important plants, cultivated by man for nutrition or for technical, in particular industrial, purposes. They may be sugar storing and/or starch-storing plants, for instance cereal species (rye, barley, oat, wheat, maize, millet, sago etc.), rice, pea, marrow pea, cassava, sugar cane, sugar beet and potato; tomato, rape, soybean, hemp, flax, sunflower, cow pea or arrowroot, fiber-forming plants (e.g. flax, hemp, cotton), oil-storing plants (e.g. rape, sunflower, soybean) and protein-storing plants (e.g. legumes, cereals, soybeans). Preferably, the invention refers to Brassicaceae species such as cabbage varieties or rape seed. The plants within the scope of the invention also include fruit trees, palms and other trees or wooden plants being of economical value such as in forestry. Moreover, the method of the invention relates to forage plants (e.g. forage and pasture grasses, such as alfalfa, clover, ryegrass) and vegetable plants (e.g. tomato, lettuce, chicory) and ornamental plants (e.g. roses, tulips, hyacinths).

According to the provisions of the invention, transgenic plants can be prepared by introducing a polynucleotide into plant cells and regenerating the transformed cells to plants by methods well known to the person skilled in the art.

Methods for the introduction of foreign genes into plants are also well known in the art. These include, for example, the transformation of plant cells or tissues with T-DNA using Agrobacteium tumefaciens or Agrobacterium rhizogenes, the fusion of protoplasts, direct gene transfer (see, e.g., EP-A 164 575), injection, electroporation, vacuum infiltration, biolistic methods like particle bombardment, pollen-mediated transformation, plant RNA virus-mediated transformation, liposome-mediated transformation, transformation using wounded or enzyme-degraded immature embryos, or wounded or enzyme-degraded embryogenic callus and other methods known in the art. The vectors used in the method of the invention may contain further functional elements, for example “left border”- and “right border”-sequences of the T-DNA of Agrobacterium which allow stable integration into the plant genome. Furthermore, methods and vectors are known to the person skilled in the art which permit the generation of marker free transgenic plants, i.e. the selectable or scorable marker gene is lost at a certain stage of plant development or plant breeding. This can be achieved by, for example co-transformation (Lyznik, Plant Mol. Biol. 13 (1989), 151-161; Peng, Plant Mol. Biol. 27 (1995), 91-104) and/or by using systems which utilize enzymes capable of promoting homologous recombination in plants (see, e.g., WO97/08331; Bayley, Plant Mol. Biol. 18 (1992), 353-361); Lloyd, Mol. Gen. Genet. 242 (1994), 653-657; Maeser, Mol. Gen. Genet. 230 (1991), 170-176; Onouchi, Nucl. Acids Res. 19 (1991), 6373-6378). Methods for the preparation of appropriate vectors are described by, e.g., Sambrook and Russell (2001), Molecular Cloning: A Laboratory Manual, CSH Press, Cold Spring Harbor, N.Y., USA.

Suitable strains of Agrobacterium tumefaciens and vectors as well as transformation of Agrobacteria and appropriate growth and selection media are well known to those skilled in the art and are described in the prior art (GV3101 (pMK9ORK), Koncz, Mol. Gen. Genet. 204 (1986), 383-396; C58C1 (pGV 3850kan), Deblaere, Nucl. Acid Res. 13 (1985), 4777; Bevan, Nucleic. Acid Res. 12(1984), 8711; Koncz, Proc. Natl. Acad. Sci. USA 86 (1989), 8467-8471; Koncz, Plant Mol. Biol. 20 (1992), 963-976; Koncz, Specialized vectors for gene tagging and expression studies. In: Plant Molecular Biology Manual Vol 2, Gelvin and Schilperoort (Eds.), Dordrecht, The Netherlands: Kluwer Academic Publ. (1994), 1-22; EP-A-120 516; Hoekema: The Binary Plant Vector System, Offsetdrukkerij Kanters B. V., Alblasserdam (1985), Chapter V, Fraley, Crit. Rev. Plant. Sci., 4, 1-46; An, EMBO J. 4 (1985), 277-287). Although the use of Agrobacterium tumefaciens is preferred in the method of the invention, other Agrobacterium strains, such as Agrobacterium rhizogenes, may be used, for example if a phenotype conferred by said strain is desired.

Methods for the transformation using biolistic methods are well known to the person skilled in the art; see, e.g., Wan, Plant Physiol. 104 (1994), 3748; Vasil, Bio/Technology 11 (1993), 1553-1558 and Christou (1996) Trends in Plant Science 1, 423-431. Microinjection can be performed as described in Potrykus and Spangenberg (eds.), Gene Transfer To Plants. Springer Verlag, Berlin, NY (1995).

The transformation of most dicotyledonous plants is possible with the methods described above. But also for the transformation of monocotyledonous plants several successful transformation techniques have been developed. These include the transformation using biolistic methods as, e.g., described above as well as protoplast transformation, electroporation of partially permeabilized cells, introduction of DNA using glass fibers, etc. Also, the transformation of monocotyledonous plants by means of Agrobacterium-based vectors has been described (Chan et al., Plant Mol. Biol. 22 (1993), 491-506; Hiei et al., Plant J. 6 (1994) 271-282; Deng et al, Science in China 33 (1990), 28-34; Wilmink et al, Plant Cell Reports 11 (1992), 76-80; May et al., Bio/Technology 13 (1995), 486-492; Conner and Dormisse, Int. J. Plant Sci. 153 (1992), 550-555; Ritchie et al. Transgenic Res. 2 (1993), 252-265). An alternative system for transforming monocotyledonous plants is the transformation by the biolistic approach (Wan and Lemaux, Plant Physiol. 104 (1994), 37-48; Vasil et al., Bio/Technology 11 (1993), 1553-1558; Ritala et al., Plant Mol. Biol. 24 (1994) 317-325; Spencer et al., Theor. Appl. Genet. 79 (1990), 625-631). The transformation of maize in particular has been repeatedly described in the literature (see for instance WO 95/06128, EP 0 513 849, EP 0 465 875, EP 29 24 35; Fromm et al, Biotechnology 8, (1990), 833-844; Gordon-Kamm et al., Plant Cell 2, (1990), 603-618; Koziel et al., Biotechnology 11 (1993), 194-200; Moroc et al., Theor. Appl. Genet. 80, (1990), 721-726). The successful transformation of other types of cereals has also been described for instance of barley (Wan and Lemaux, supra; Ritala et al., supra, Krens et al., Nature 296 (1982), 72-74), wheat (Nehra et al., Plant J. 5 (1994), 285-297) and rice.

The resulting transformed plant cell can then be used to regenerate a transformed plant in a manner known by a skilled person.

In addition, the present invention relates to transgenic plants which show an increased activity of the polypeptide encoded by the polynucleotide the invention compared to a corresponding wild-type plant.

The term “increased activity” refers to a significant increase of the beta-glucosidase activity of the polypeptide of the invention in the transgenic plant compared to a corresponding wild-type plant. Preferably, said activity is increased in the transgenic plant by at least 10%, preferably by at least 20%, more preferably by at least 50%, and even more preferred by at least 100% as compared to the corresponding wild-type plant. Beta-glucosidase activity may be determined in enzyme assays using a preparation from a plant sample. In particular, this assay is specific enough to exclude any other beta-glucosidase activity present in the plant. Advantageously, a substrate compound is used for such assays for which the polypeptide of the invention is specific and which can be detected by suitable methods known in the art. However, an increase of the activity of the polypeptide of the invention may also be inferred from a significant increase of the amount of corresponding transcript and/or protein present in the transgenic plant. Preferentially, transgenic plants having an increased activity of the polypeptide of the invention may be characterized by an increase of the amount of transcript corresponding to the polynucleotide of the invention by at least 20%, preferably at least 50% and more preferably at least 100% as compared to the corresponding wild-type plant. Likewise, it is preferred that transgenic plants having an increased activity of the polypeptide of the invention may be characterized by an increase of the protein amount of the polypeptide of the invention by at least 20%, preferably at least 50% and more preferably at least 100% as compared to the corresponding wild-type plant.

Corresponding increases of the activity of the polypeptide of the invention may for instance be achieved by expressing said polynucleotide in cells of a transgenic plant from a heterologous construct for example as described above. However, the state of the art provides further methods for achieving a corresponding increased activity. For example, the endogenous gene encoding the beta-glucosidase of the invention may be modified accordingly at its natural location, e.g. by homologous recombination. In particular, the promoter of this gene can for instance be altered in a way that promoter activity is enhanced. In the alternative, the coding region of the gene can be modified so that the encoded polypeptide shows an increased activity, e.g. by specifically substituting amino acid residues in the catalytically active domain of the polypeptide. Applicable homologous recombination techniques (also known as “in vivo mutagenesis”) are known to the person skilled in the art and are described in the literature. One such technique involves the use of a hybrid RNA-DNA oligonucleotide (“chimeroplast”) which is introduced into cells by transformation (TIBTECH 15 (1997), 441-447; WO95/15972; Kren, Hepatology 25 (1997),1462-1468; Cole-Strauss, Science 273 (1996), 1386-1389). Thereby, part of the DNA component of the RNA-DNA oligonucleotide is homologous with the target gene sequence, however, displays in comparison to this sequence a mutation or a heterologous region which is surrounded by the homologous regions. The term “heterologous region” refers to any sequence that can be introduced and which is different from that to be modified. By means of base pairing of the homologous regions with the target sequence followed by a homologous recombination, the mutation or the heterologous region contained in the DNA component of the RNA-DNA oligonucleotide can be transferred to the corresponding gene. By means of in vivo mutagenesis, any part of the gene encoding the beta-glucosidase of the invention can be modified as long as it results in an increase of the activity of this protein.

In a preferred embodiment, the above-described transgenic plants show, upon an increased activity of the protein encoded by the polynucleotide of the invention, an increased resistance against a plant pathogen to which a corresponding wild-type plant is susceptible.

The term “increased resistance” may refer both to an enhancement of a resistance already present in the wild-type plant and to the establishment of a resistance that is not present in the wild-type plant.

Preferably, these transgenic plants contain a polynucleotide as defined above, i.e. a polynucleotide or a recombinant nucleic acid molecule that is introduced in a plant cell and the presence of which in the genome of said plant preferably leads to an increased activity of the beta-glucosidase of the invention, stably integrated into the genome.

In a further aspect, the present invention relates to transgenic plants which, upon the presence of a suitable nucleic acid molecule in the genome of its cells, show a reduced activity of the beta-glucosidase of the invention.

Such transgenic plants may be useful objects for studying the mechanism of non-host resistance and the role the polypeptide of the invention plays in that mechanism. The above explanations concerning techniques for producing transgenic plants and plant cells as well as suitable transformation techniques and vectors mentioned in connection with the transgenic plants having an increased activity of the beta-glucosidase of the invention may be likewise applied in the present embodiment.

Methods for specifically reducing the activity of a protein in plant cells by the introduction of nucleic acid molecules are exhaustively and widely described in the literature and are known to the person skilled in the art. These include but are not limited to antisense, ribozyme, co-suppression, RNA interference, expression of dominant negative mutants, antibody expression and in vitro mutagenesis approaches.

The term “presence of a suitable nucleic acid molecule” as used herein refers to any foreign nucleic acid molecule that is present in cells of a transgenic plant produced in accordance with the invention but absent from the cells of the corresponding source plant. Thereby encompassed are nucleic acid molecules, e.g. gene sequences, which differ from the corresponding nucleic acid molecule in the source plant cell by at least one mutation (substitution, insertion, deletion, etc. of at least one nucleotide), wherein such a mutation inhibits the expression of the affected gene or reduces the activity of the gene product. Furthermore encompassed by the term “foreign” are nucleic acid molecules which are homologous with respect to the source plant cell but are situated in a different chromosomal location or differ, e.g., by way of a reversed orientation for instance with respect to the promoter.

In principle, the nucleic acid molecule to be introduced in accordance with the present embodiment may be of any conceivable origin, e.g. eukaryotic or prokaryotic. It may be from any organism which comprises such molecules. Furthermore, it may be synthetic or derived from naturally occurring molecules by, e.g., modification of its sequence, i.e. it may be a variant or derivative of a naturally occurring molecule. Such variants and derivatives include but are not limited to molecules derived from naturally occurring molecules by addition, deletion, mutation of one or more nucleotides or by recombination. It is, e.g., possible to change the sequence of a naturally occurring molecule so as to match the preferred codon usage of plants, in particular of those plants in which the nucleic acid molecule shall be expressed.

It is furthermore preferred that the nucleic acid molecule introduced into a plant cell in accordance with the present embodiment has to be expressed in the transgenic plant in order to exert the reducing effect upon beta-glucosidase activity. The term “expressed” means for such a nucleic acid molecule that it is at least transcribed, and for some embodiments also translated into a protein, in at least some of the cells of the plant. Preferred examples of such nucleic acid molecules relate to those embodiments of the transgenic plants of the invention wherein said reduced beta-glucosidase activity is achieved by an antisense, co-suppression, ribozyme or RNA interference effect or by the expression of antibodies or other suitable (poly)peptides capable of specifically reducing said acitvity or by the expression of a dominant-negative mutant. These methods are further explained in the following.

Accordingly, the use of nucleic acid molecules encoding an antisense RNA which is complementary to transcripts of a gene encoding a plant beta-glucosidase of the invention is a preferred embodiment of the present invention. Thereby, complementarity does not signify that the encoded RNA has to be 100% complementary. A low degree of complementarity may be sufficient as long as it is high enough to inhibit the expression of such a beta-glucosidase protein upon expression of said RNA in plant cells. The transcribed RNA is preferably at least 90% and most preferably at least 95% complementary to the transcript of the nucleic acid molecule encoding PEN2. In order to cause an antisense effect during the transcription in plant cells such RNA molecules have a length of at least 15 bp, preferably a length of more than 100 bp and most preferably a length or more than 500 bp, however, usually less than 2000 bp, preferably shorter than 1500 bp. Exemplary methods for achieving an antisense effect in plants are for instance described by Müller-Röber (EMBO J. 11 (1992), 1229-1238), Landschütze (EMBO J. 14 (1995), 660-666), D'Aoust (Plant Cell 11 (1999), 2407-2418) and Keller (Plant J. 19 (1999), 131-141) and are herewith incorporated in the description of the present invention. Likewise, an antisense effect may also be achieved by applying a triple-helix approach, whereby a nucleic acid molecule complementary to a region of the gene, encoding the relevant beta-glucosidase, designed according to the principles for instance laid down in Lee (Nucl. Acids Res. 6 (1979), 3073); Cooney (Science 241 (1998), 456) or Dervan (Science 251 (1991), 1360) may inhibit its transcription.

A similar effect as with antisense techniques can be achieved by producing transgenic plants expressing suitable constructs in order to mediate an RNA interference (RNAi) effect. Thereby, the formation of double-stranded RNA leads to an inhibition of gene expression in a sequence-specific fashion. More specifically, in RNAi constructs, a sense portion comprising the coding region of the gene to be inactivated (or a part thereof, with or without non-translated region) is followed by a corresponding antisense sequence portion. Between both portions, an intron not necessarily originating from the same gene may be inserted. After transcription, RNAi constructs form typical hairpin structures. In accordance with the teachings of the present invention, the RNAi technique may be carried out as described by Smith (Nature 407 (2000), 319-320) or Marx (Science 288 (2000), 1370-1372).

Also DNA molecules can be employed which, during expression in plant cells, lead to the synthesis of an RNA which reduces the expression of the gene encoding the beta-glucosidase of the invention in the plant cells due to a co-suppression effect. The principle of co-suppression as well as the production of corresponding DNA sequences is precisely described, for example, in WO 90/12084. Such DNA molecules preferably encode an RNA having a high degree of homology to transcripts of the target gene. It is, however, not absolutely necessary that the coding RNA is translatable into a protein. The principle of the co-suppression effect is known to the person skilled in the art and is, for example, described in Jorgensen, Trends Biotechnol. 8 (1990), 340-344; Niebel, Curr. Top. Microbiol. Immunol. 197 (1995), 91-103; Flavell, Curr. Top. Microbiol. Immunol. 197 (1995), 43-36; Palaqui and Vaucheret, Plant. Mol. Biol. 29 (1995), 149-159; Vaucheret, Mol. Gen. Genet. 248 (1995), 311-317; de Borne, Mol. Gen. Genet. 243 (1994), 613-621 and in other sources.

Likewise, DNA molecules encoding an RNA molecule with ribozyme activity which specifically cleaves transcripts of a gene encoding the relevant beta-glucosidase can be used. Ribozymes are catalytically active RNA molecules capable of cleaving RNA molecules and specific target sequences. By means of recombinant DNA techniques, it is possible to alter the specificity of ribozymes. There are various classes of ribozymes. For practical applications aiming at the specific cleavage of the transcript of a certain gene, use is preferably made of representatives of the group of ribozymes belonging to the group I intron ribozyme type or of those ribozymes exhibiting the so-called “hammerhead” motif as a characteristic feature. The specific recognition of the target RNA molecule may be modified by altering the sequences flanking this motif. By base pairing with sequences in the target molecule, these sequences determine the position at which the catalytic reaction and therefore the cleavage of the target molecule takes place. Since the sequence requirements for an efficient cleavage are low, it is in principle possible to develop specific ribozymes for practically each desired RNA molecule. In order to produce DNA molecules encoding a ribozyme which specifically cleaves transcripts of a gene encoding the relevant beta-glucosidase, for example a DNA sequence encoding a catalytic domain of a ribozyme is bilaterally linked with DNA sequences which are complementary to sequences encoding the target protein. Sequences encoding the catalytic domain may for example be the catalytic domain of the satellite DNA of the SCMo virus (Davies, Virology 177 (1990), 216-224 and Steinecke, EMBO J. 11 (1992), 1525-1530) or that of the satellite DNA of the TobR virus (Haseloff and Gerlach, Nature 334 (1988), 585-591). The expression of ribozymes in order to decrease the activity of certain proteins in cells is known to the person skilled in the art and is, for example, described in EP-B1 0 321 201. The expression of ribozymes in plant cells is for example described in Feyter (Mol. Gen. Genet. 250 (1996), 329-338).

Furthermore, nucleic acid molecules encoding antibodies specifically recognizing the relevant beta-glucosidase in a plant, i.e. specific fragments or epitopes of such a protein, can be used for inhibiting the activity of this protein. These antibodies can be monoclonal antibodies, polyclonal antibodies or synthetic antibodies as well as fragments of antibodies, such as Fab, Fv or scFv fragments etc. Monoclonal antibodies can be prepared, for example, by the techniques as originally described in Köhler and Milstein (Nature 256 (1975), 495) and Galfré (Meth. Enzymol. 73 (1981) 3), which comprise the fusion of mouse myeloma cells to spleen cells derived from immunized mammals. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. Expression of antibodies or antibody-like molecules in plants can be achieved by methods well known in the art, for example, full-size antibodies (Düring, Plant. Mol. Biol. 15 (1990), 281-293; Hiatt, Nature 342 (1989), 469-470; Voss, Mol. Breeding 1 (1995), 39-50), Fab-fragments (De Neve, Transgenic Res. 2 (1993), 227-237), scFvs (Owen, Bio/Technology 10 (1992), 790-794; Zimmermann, Mol. Breeding 4 (1998), 369-379; Tavladoraki, Nature 366 (1993), 469-472; Artsaenko, Plant J. 8 (1995), 745-750) and variable heavy chain domains (Benvenuto, Plant Mol. Biol. 17 (1991), 865-874) have been successfully expressed in tobacco, potato (Schouten, FEBS Lett. 415 (1997), 235-241) or Arabidopsis, reaching expression levels as high as 6.8% of the total protein (Fiedler, Immunotechnology 3 (1997), 205-216).

Moreover, also nucleic acid molecules encoding peptides or polypeptides capable of reducing the activity of the relevant ABC transporter other than antibodies can be used in the present context. Examples of suitable peptides or polypeptides that can be constructed in order to achieve the intended purpose can be taken from the prior art and include, for instance, binding proteins such as lectins.

In addition, nucleic acid molecules encoding a mutant form of the relevant beta-glucosidase can be used to interfere with the activity of the wild-type protein. Such a mutant form preferably has lost its biological activity, e.g. its hydrolytic activity on glycosidic bonds, and may be derived from the corresponding wild-type protein by way of amino acid deletion(s), substitution(s), and/or additions in the amino acid sequence of the protein. Mutant forms of such proteins may show, in addition to the loss of the hydrolytic activity, an increased substrate affinity and/or an elevated stability in the cell, for instance, due to the incorporation of amino acids that stabilize proteins in the cellular environment. These mutant forms may be naturally occurring or, as preferred, genetically engineered mutants.

In another preferred embodiment, the nucleic acid molecule, the presence of which in the genome of a plant cell leads to a reduction of the activity of the beta-glucosidase of the invention, does not require its expression to exert its reducing effect on beta-glucosidase activity. Correspondingly, preferred examples relate to methods wherein said reduced beta-glucosidase activity is achieved by in vivo mutagenesis or by the insertion of a heterologous DNA sequence in the gene encoding the beta-glucosidase of the invention.

The term “in vivo mutagenesis”, relates to methods where the sequence of the gene encoding the relevant beta-glucosidase is modified at its natural chromosomal location such as for instance by techniques applying homologous recombination. This may be achieved by using a hybrid RNA-DNA oligonucleotide (“chimeroplast”) which is introduced into cells by transformation (TIBTECH 15 (1997), 441-447; WO95/15972; Kren, Hepatology 25 (1997), 1462-1468; Cole-Strauss, Science 273 (1996), 1386-1389). Part of the DNA component of the RNA-DNA oligonucleotide is homologous to the target ABC transporter gene sequence, however, displays in comparison to this sequence a mutation or a heterologous region which is surrounded by the homologous regions. The term “heterologuous region” corresponds to any sequence that can be introduced and encompasses, for instance, also sequences from the same beta-glucosidase gene but from a different site than that which is to be mutagenized. By means of base pairing of the homologous regions with the target sequence followed by a homologous recombination, the mutation or the heterologous region contained in the DNA component of the RNA-DNA oligonucleotide can be transferred to the corresponding gene of the plant cell. By means of in vivo mutagenesis, any part of the gene encoding the beta-glucosidase can be modified as long as it results in a decrease of the beta-glucosidase activity. Thus, in vivo mutagenesis can for instance concern, the promoter, e.g. the RNA polymerase binding site, as well as the coding region, in particular those parts encoding the substrate binding site or the catalytically active site or a signal sequence directing the protein to the appropriate cellular compartment.

The term “heterologous DNA sequence” refers to any DNA sequences which can be inserted into the target gene via appropriate techniques other than those described above in connection with in vivo mutagenesis. The insertion of such a heterologous DNA sequence may be accompanied by other mutations in the target gene such as the deletion, inversion or rearrangement of the sequence located at the insertion site. This embodiment of the invention includes that the introduction of a nucleic acid molecule leads to the generation of a pool, i.e. a plurality, of transgenic plants in the genome of which the nucleic acid molecule, i.e. the heterologous DNA sequence, is randomly spread over various chromosomal locations, and that this generation of transgenic plants is followed by selecting those transgenic plants out of the pool which show the desired genotype, i.e. an inactivating insertion in the relevant beta-glucosidase gene and/or the desired phenotype, i.e. a reduced beta-glucosidase activity and/or reduced non-host resistance as for example described for the pen2 mutant plants (see Examples 2 and 3).

Suitable heterologous DNA sequences that can be taken for such an approach are described in the literature and include, for instance vector sequences capable of self-integration into the host genome or mobile genetic elements. Particularly preferred in this regard are T-DNA or transposons which are well-known to the person skilled in the art from so-called tagging experiments used for randomly knocking out genes in plants. The production of such pools of transgenic plants can for example be carried out as described in Jeon (Plant J. 22 (2000), 561-570) or Parinov (Curr. Op. Biotechnol. 11 (2000), 157-161).

Another example of insertional mutations that may result in gene silencing includes the duplication of promoter sequences which may lead to a methylation and thereby an inactivation of the promoter (Morel, Current Biology 10 (2000), 1591-1594).

Furthermore, it is immediately evident to the person skilled in the art that the above-described approaches, such as antisense, ribozyme, co-suppression, in-vivo mutagenesis, RNAi, expression of antibodies, other suitable peptides or polypeptides or dominant-negative mutants and the insertion of heterologous DNA sequences, can also be used for the reduction of the expression of genes that encode a regulatory protein such as a transcription factor, that controls the expression of the relevant beta-glucosidase or, e.g., proteins that are necessary for the beta-glucosidase to become active.

It is also evident from the disclosure of the present invention that any combination of the above-identified approaches can be used for the generation of transgenic plants, which due to the one or more of the above-described nucleic acid molecules in their cells, display a reduced activity of the relevant beta-glucosidase compared to corresponding source plants. Such combinations can be made, e.g., by (co-) transformation of corresponding nucleic acid molecules into the plant cell, plant tissue or plant or by crossing transgenic or mutant plants that have been generated according to different techniques. Likewise, the transgenic plants of the present invention showing a reduced activity of the beta-glucosidase of the invention can be crossed with plants, e.g. transgenic plants, having other desired traits.

The invention also relates to propagation material of the transgenic plants of the invention comprising plant cells according to the invention. The term “propagation material” comprises those components or parts of the plant which are suitable to produce offspring vegetatively or generatively. Suitable means for vegetative propagation are for instance cuttings, callus cultures, rhizomes or tubers. Other propagation material includes for instance fruits, seeds, seedlings, protoplasts, cell cultures etc. The preferred propagation materials are tubers and seeds.

The invention also relates to harvestable parts of the plants of the invention such as, for instance, fruits, seeds, tubers, rootstocks, leaves or flowers.

Corresponding to the above explanations, the invention furthermore relates to a method for conferring pathogen resistance or increased pathogen resistance to a plant comprising the step of providing a transgenic plant in which the activity of the polypeptide encoded by the above-described polynucleotide of the invention is increased.

In a further aspect, the present invention relates to methods for identifying a compound that is hydrolyzed by the polypeptide of the invention. Some of the methods may comprise the step of contacting a candidate compound with an effector molecule (such as said polypeptide). This step may be accomplished by adding a sample containing said candidate compound or a plurality of candidate compounds to the assay mixture. If such a sample or plurality of compounds is identified in one of the methods to contain a compound of interest, then it is either possible to isolate the compound from the original sample or one can further subdivide the original sample, for example, if it consists of a plurality of different compounds, so as to reduce the number of different substances per sample and repeat the method with the subdivisions of the original sample. Depending on the complexity of the samples, the steps described herein can be performed several times, preferably until the sample identified according to the method of the invention only comprises a limited number of or only one substance(s). Preferably said sample comprises substances of similar chemical and/or physical properties, and most preferably said substances are identical.

Furthermore, a compound identified by said method(s) may be known in the art but hitherto not known to be a substrate for the polypeptide of the invention, preferably it was not known that said compound or a hydrolysis product thereof is capable of increasing resistance against pathogens in plants.

Accordingly, in one aspect, the present invention relates to a method for identifying a compound that is hydrolyzed by a polypeptide encoded by the above-described polynucleotide of the invention comprising the steps of
(a) contacting a candidate compound with said polypeptide under conditions where said polypeptide is active; and
(b) determining whether said candidate compound is hydrolyzed by said polypeptide.
   The polypeptide to be used in this method can be provided as described above, for example from a natural source from which the polypeptide preferably is purified by applying suitable techniques known in the art, by chemical synthesis or by recombinant expression. Preferentially, the polypeptide is expressed in a prokaryotic host, preferably a bacterial cell such as an Escherichia coli cell. The method for identifying a substrate compound may be performed using the cell culture medium or the supernatant thereof into which the polypeptide has been secreted. Also, the polypeptide may be purified to a degree which is necessary by conventional techniques in order to obtain significant results in said method. In a preferred embodiment, the polypeptide may be used in an immobilized form, i.e. directly or indirectly via the linkage over suitable intermediate molecules (e.g. peptide linkers, antibodies and the like) attached to a solid support. In a further preferred embodiment, the method for identifying a substrate compound may be set up in a high-throughput fashion. Suitable strategies in order to achieve high-throughput scale for enzyme assays are known to a skilled person and are described in the literature. In the prior art, several approaches are described for overexpression of a polypeptide and activity determination that can be adapted for use in the present method of the invention such as the approaches described by Minami (Plant Cell Physiol. 41 (2000), 218-225), Chen (Protein Expression and Purification 17 (1999), 414-421) and Dharmawardhana (Plant Mol. Biol. 40 (1999), 365-372).

In addition, the present invention relates to a method for identifying a compound that is hydrolyzed by a polypeptide encoded by the above-described polynucleotide of the invention comprising the steps of
(a) providing a 3-dimensional structure model of said polypeptide; and
(b) determining the structure of a substrate that fits into the 3-dimensional structure model of (a).
   Three-dimensional structure models of the polypeptide of the invention may be provided according to appropriate techniques known to a person skilled in the art and described in the literature. Conventionally, three-dimensional data on protein structure can be obtained from x-ray crystallography. For the purposes of the present invention, crystallization, structure determination and data processing and representation in a three-dimensional model may be carried out as described in Czjzek (Proc. Natl. Acad. Sci. USA 97 (2000), 13555-13560). In this publication, results are described on x-ray crystallography of an inactive mutant of the maize beta-glucosidase Glu1 and the structural elements that determine substrate specificity of the natural substrate DIMBOAGlc as well as analogs of this compound. Therein, it was confirmed that, for beta-glucosidases, specificity is determined by the aglycone portion of the substrate. Other instructive examples for corresponding approaches are described in Czjzek (Biochem. J. 354 (2001), 37-46) and Burmeister (Structure 5 (1997), 663-675).

For obtaining information on the structure of the substrate via three-dimensional structure data, the wild-type polypeptide, mutants thereof such as catalytically inactive variants or fragments bearing the substrate-binding portion of the polypeptide can be used for crystallization.

In yet another embodiment, the invention relates to a method for identifying a compound that is hydrolyzed by a polypeptide encoded by the above-described polynucleotide of the invention comprising the steps of
(a) providing a mutant protein of said polypeptide the catalytic activity of which is abolished without loosing substrate binding activity;
(b) contacting a candidate compound with said mutant protein; and
(c) determining whether the candidate compound is bound by said mutant protein.

This method is based on the assumption that the substrate compound to be identified is characterized by its ability to be bound by the polypeptide of the invention. Thus, by identifying compounds that are bound by said polypeptide, one may restrict the scope of potential substrate molecules to a high degree, if not to the actual substrate molecule itself. The technique applied in this method is also known as “substrate trap”. For instance, Flint (Proc. Natl. Acad. Sci. USA 95 (1997), 1680-1685) applied substrate-trapping in order to identify physiological substrates of protein tyrosine phosphatases.

The provision of mutant proteins the catalytic activity of which is abolished without loosing substrate binding activity can be accomplished based on information on the amino acid residues that are known to be necessary for catalytic activity in family 1 beta-glucosidases. Such information is available from the prior art. For instance, the glutamic acid residues (E) in the conserved family 1 beta-glucosidase motifs TFNEP and (I/V)TENG (SEQ ID NOs:3 and 4) are known to be involved in the catalytic reactions and are therefore preferred targets for producing catalytically inactive mutants of the polypeptide of the invention. Czjzek (Proc. Natl. Acad. Sci. USA 97 (2000), 13555-13560) produced such a mutant by substituting the glutamic acid residue at position 191 by an aspartate residue (E191D). Accordingly, such a mutant may be produced starting from the polypeptide of the invention by substituting the amino acid residue corresponding to the glutamate residue at position 183 of SEQ ID NO: 2 by, e.g. an aspartate residue.

Inactivation of the polypeptide of the invention may be carried out according to suitable techniques known in the prior art and comprise recombinant methods as well as post-translational, for example chemical modifications. Recombinant methods include the introduction of mutations such as substitutions, additions, deletions, inversions or transversions into the amino acid sequence of the polypeptide as they are for example described in Sambrook and Russell (2001), Molecular Cloning: A Laboratory Manual, CSH Press, Cold Spring Harbor, N.Y., USA. These may for instance be accomplished by in vitro mutagenesis, e.g. based on PCR, or in vivo mutagenesis. In a preferred embodiment of the present method, the polypeptide may be used in an immobilized form, i.e. directly or indirectly via the linkage over suitable intermediate molecules (e.g. peptide linkers, antibodies and the like) attached to a solid support.

The candidate compounds to be tested in the present method can in principle be taken from any source that can be expected to contain potential beta-glucosidase substrates. Plant beta-glucosidases are for example known to be involved in the defence against pests, in lignification and in cell wall catabolism. Thus, preferably candidate compounds may be taken from corresponding sources, i.e. plant pathogens (e.g. elicitors), lignin precursors or cell wall components or precursors thereof. As it has been shown in Example 4, the pen2 mutant plants have an altered cell wall composition as compared to corresponding wild-type plants. Thus, it is particularly preferred to take the candidate compounds for use in the present method from the cell wall or cell wall precursor substances, preferably from the plant species from which the polypeptide used in the method is derived. Practically, compound libraries that are routinely used for screenings may be of use herein.

Contacting a candidate compound with the mutant protein should be carried out under conditions which allow binding of the substrate compound by the mutant protein. Preferably, the mutant protein is contacted with a plurality of candidate compounds. After the contacting step, the unbound candidate compounds can be removed from the assay mixture and subsequently the compound bound by the mutant protein, if any, can be examined in order to determine its identity. For this purpose, advantageously, the bound compound is released from the mutant protein prior to determination by taking appropriate measures. Determination of the compound's identity can be done using according techniques known in the prior art such as thin layer chromatography (TLC), capillary electrophoresis (CE), gas chromatography (GC), mass spectrometry (MS), liquid chromatography (LC), NMR or FT-IR or combinations thereof such as GC/IMS or LC/IMS. Likewise, whether the candidate compound is bound by the mutant protein may be determined by removing any unbound or non-specifically bound candidate compound contacted with the mutant protein from the assay mixture, followed by changing the assay conditions such that any specifically bound compound is released from the mutant protein. Thereby, it is possible to determine whether a compound is released and thereby determining whether the candidate compound is bound by the mutant protein and, optionally and in case there has been bound a compound, its identity may subsequently be identified using suitable techniques.

Moreover, it is evident to a person skilled in the art that the methods for identifying a compound that is hydrolyzed by a polypeptide of the invention as they are explained above can be combined. Thus, it is for instance possible in a first stage to screen a large set of compounds, possibly also pools of compounds, in substrate trap experiments or by obtaining insights into the molecular structure of potential substrate compounds through the use of 3-dimensional structure data. Then, in a second stage of screening, pre-selected candidate compounds can be subjected to hydrolysis assays using catalytically active polypeptide preparations.

In a preferred embodiment, the above-outlined methods for identifying a compound that is hydrolyzed by the polypeptide of the invention furthermore comprise the step of determining whether the identified compound or, as it is preferred, a hydrolysis product thereof is capable of inducing or enhancing a defence response against a pathogen in a plant.

In this additional step within the process of identifying a substrate compound to the polypeptide of the invention, the compound that is identified by any one of the above-outlined methods or combinations thereof is further tested for the ability to activate defence against a pathogen in a plant. It is to be noted that this requires that the plant used expresses the polypeptide of the invention that is specific for the substrate compound used since it is contemplated that the aglucone product of said compound is the form active in plant defence. Therefore, in a preferred embodiment, plants are used in the present method that express the specific polypeptide of the invention, either naturally or, which is especially preferred, because they are transgenic plants transformed with a construct expressing said polypeptide.

In any case or if the respective plant does not express this polypeptide, one or more hydrolysis products of the compound may be used in order to test for defence-activating activity. The term “hydrolysis product” as used throughout the present application refers to the products that result from a hydrolysis reaction acting on a substrate molecule catalyzed by the polypeptide of the invention. For example, the hydrolysis product may be the aglycone portion of a substrate which is a conjugate compound. Aglycone portions of beta-glucosidase substrates are known to be diverse with regard to their chemical nature and their biological function. Such aglycones may for instance comprise plant hormones, flavonoids or cyanogenic compounds. The aglycon product of the hydrolyzed substrates can serve a multitude of functions including growth and development (Selmar et al., 1987; Brzobohaty et al., 1993; Dietz et al., 2000), cell wall catabolism (Leah et al., 1995; Gerardi et al., 2001), lignification (Dharmawardhana et al., 1995 and 1999), and defense (Zheng and Poulton, 1995; Rsak et al., 2000). However, if for example the substrate of the polypeptide of the invention is an oligo- or polysaccharide, both hydrolysis products may be carbohydrates or at least compounds comprising glycosidic portions. For instance Gerardi (Plant Science 160 (2001), 795-805) describe beta-glucosidases that can efficiently cleave cell wall oligo- or polysaccharides.

Hydrolysis products can be produced by the use of the polypeptide of the invention and optionally purified according to methods described in the prior art. However, once the identity of the products is known by way of having applied the above-described method(s) for identifying a substrate compound of the polypeptide of the invention, the products may likewise be provided by other methods than by using the polypeptide of the invention which are known in the art such as by isolation from natural sources or by chemical synthesis.

In a preferred embodiment of the method for identifying substrate compounds, mutant plants are used, wherein the gene encoding the polypeptide of the invention is inactive such as in the pen2 mutant described in connection with the present invention.

For the purpose of testing whether a defence response against a pathogen is induced or enhanced, test plants or parts thereof are inoculated with a corresponding pathogen in the presence and in the absence of the compound or a hydrolysis product thereof. Instead of inoculation, the plant or part thereof may alternatively be treated with an elicitor if appropriate. Susceptibility to the pathogen may then be examined according to methods described in the prior art or as exemplified in the appended Examples. A reduction of susceptibility due to the presence of the compound or a hydrolysis product thereof is indicative for the ability of said compound or hydrolysis product thereof to induce or enhance a defence response against a pathogen in a plant. The term “inducing a defence response” refers to the situation where the plant does normally not show a significant defence reaction to the respective pathogen, for instance when a compatible interaction takes place with the pathogen. On the other hand, “enhancing a defence response” refers to the situation where the plant normally shows a defence response, e.g. in an incompatible interaction with the pathogen or as part of a non-host resistance, but the presence of the compound or hydrolysis product thereof results in an enhancement of the defence reaction(s) with the effect that the plant is less susceptible to the pathogen. “Less susceptible” may mean in this context a reduction of successful colonization of the pathogen into plant tissue by at least 10%, preferably by at least 20%, more preferably by at least 50% and even more preferably by at least 80%. The term “successful colonisation” refers to colonization of the pathogen on or in the plant which are stable, i.e. in contrast to colonization which cease after a certain while due to lacking nutrition for the pathogen. With regard to fungal pathogens, like for instance powdery mildew, such a successful colonisation is for instance apparent from the formation of a haustorium and of secondary hyphae.

In yet another embodiment, the present invention relates to a method for preparing a plant protection composition comprising the steps of at least one of the methods for identifying a compound that is hydrolyzed by a polypeptide of the invention described above and furthermore the step of formulating the identified compound or a hydrolysis product thereof preferably being capable of inducing or enhancing a defense response against a pathogen in a plant in a form suitable for administering to plants.

The compound identified according to the above-described method(s) or an analog or derivative thereof may be further formulated in a form suitable for the application in plant cultivation. For example, it can be combined with a agriculturally acceptable carrier known in the art. The plant protection composition can be prepared by employing one or more of the above-described methods for identifying the compound and synthesizing the compound or a hydrolysis product thereof in an amount sufficient for use in agriculture. Thus, the term “formulating” also encompasses synthesizing the compound so-identified or a hydrolysis product thereof or an analog or derivative thereof. “Analogs or derivatives” refer to compounds that show substantially the same activity with respect to the potential to increase resistance in plants as the originally identified compound or a hydrolysis product thereof and that are immediately recognizable by a person skilled in the art in the field of agrochemicals once he/she is aware of the originally identified compound or a hydrolysis product thereof.

In the plant protection composition of the invention, the compound identified by the above-described method or a hydrolysis product thereof may be formulated by conventional means commonly used for the application of, for example, herbicides and pesticides or agents capable of inducing systemic acquired resistance (SAR). For example, certain additives known to those skilled in the art such as stabilizers or substances which facilitate the uptake by the plant cell, plant tissue or plant may be used as for example harpins, elicitins, salicylic acid (SA), benzol(1,2,3)thiadiazole-7-carbothioic acid (BTH), 2,6-dichloro isonicotinic acid (INA), jasmonic acid (JA) or methyljasmonate.

Accordingly, the present invention furthermore relates to a compound, a hydrolysis product thereof or a plant protection composition that is identified or obtained by any one of the corresponding methods described above.

In addition, the present invention relates to a kit comprising the polynucleotide, the recombinant nucleic acid molecule, the vector, the polypeptide or the antibody of the invention or the compound that is hydrolyzed by the polypeptide of the invention or a hydrolysis product thereof.

The kit of the invention may contain further ingredients such as selection markers and components for selective media suitable for the generation of transgenic plant cells, plant tissue or plants. Furthermore, the kit may include materials such as buffers and candidate substrates for carrying out one of the above-described methods for identifying compounds that are hydrolyzed by the polypeptide of the invention. Thus, the kit may for example contain nucleic acid molecules useful as probes for identifying an ortholog of the Arabidopsis beta-glucosidase gene disclosed herein in other plant species. Furthermore, the kit may contain materials useful for expressing the polypeptide and/or for crystallizing it. The kit of the invention may advantageously be used for carrying out the methods of the invention and could be, inter alia, employed in a variety of applications referred to herein, e.g., as research tool. The parts of the kit of the invention can be packaged individually in vials or in combination in containers or multicontainer units. Manufacture of the kit follows preferably standard procedures which are known to the person skilled in the art.

The kit or its ingredients according to the invention can be used, for example, for any of the above described methods for identifying compounds that enhance or establish non-host resistance in plants. The kit of the invention and its ingredients are expected to be very useful in breeding new varieties of, for example, plants which display improved properties such as disease resistance.

It is also immediately evident to the person skilled in the art that the polynucleotides, recombinant nucleic acid molecules and vectors of the present invention can be employed to produce transgenic plants with a desired trait (see for review TIPTEC Plant Product & Crop Biotechnology 13 (1995), 312-397) comprising (i) insect resistance (Vaek, Plant Cell 5 (1987), 159-169), (ii) virus resistance (Powell, Science 232 (1986), 738-743; Pappu, World Journal of Microbiology & Biotechnology 11 (1995), 426-437; Lawson, Phytopathology 86 (1996), 56 suppl.), (iii) resistance to bacteria, insects and fungi (Duering, Molecular Breeding 2 (1996), 297-305; Strittmatter, Bio/Technology 13 (1995), 1085-1089; Estruch, Nature Biotechnology 15 (1997), 137-141), or (iv) as a genetic marker useful in breeding plants with an improved resistance to pathogens.

In a further embodiment, the present invention relates to the use of the polynucleotide, of the recombinant nucleic acid molecule, of the vector, of the host cell, of the polypeptide, of the antibody or of the transgenic plant of the invention, said matters being described above in detail, for identifying a compound that is hydrolyzed by a polypeptide encoded by said polynucleotide.

For this use, preferably, any one of the above-mentioned methods for identifying such a compound may be applied. However, the present invention additionally encompasses the corresponding use of the matters given above by applying other methods that can be envisaged.

Another embodiment of the present invention relates to the use of the polynucleotide, of the recombinant nucleic acid molecule, of the vector, of the host cell, of the polypeptide, of the antibody or of the transgenic plant of the invention, said matters being described above in detail, for the preparation of a plant protection composition.

Also within the scope of the invention is the use of the of the polynucleotide, of the recombinant nucleic acid molecule, of the vector, of the host cell, of the polypeptide, of the antibody or of the transgenic plant of the invention, said matters being described above in detail, for establishing or enhancing a pathogen resistance in plants.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.google.de. An overview of patent information in biotechnology and a survey of relevant sources of patent information useful for retrospective searching and for current awareness is given in Berks, TIBTECH 12 (1994), 352-364.

Furthermore, the term “and/or” when occurring herein includes the meaning of “and”, “or” and “all or any other combination of the elements connected by said term”.

The present invention is further described by reference to the following non-limiting figures and examples.

The Figures show:

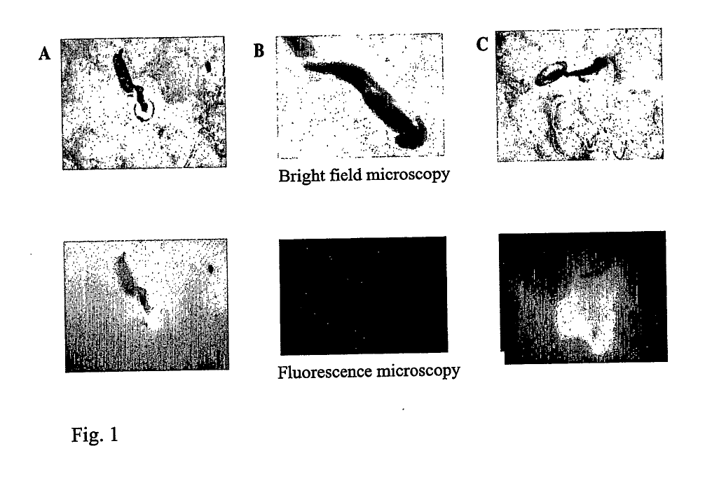

FIG. 1 shows on a microscopic scale typical events during the non-host interaction between Arabidopsis thaliana and Blumeria graminis f. sp. hordei (Bgh).
A. Bgh spores efficiently adhere, germinate and develop appressoria on Arabidopsis leaves. Most aborted penetration attempts correlate with a local cell wall remodeling (24 h p. inoc.).
B. Successful penetration and haustorium initial formation. Encasement in callose terminates the fungal development (48 h p. inoc.).
C. Thickening of the complete cell wall and typical whole cell autofluorescence in a successfully penetrated epidermis cell (72 h p. inoc.).

FIG. 2 illustrates the mutant phenotype of pen2 5 d p. inoc. Fluorescence microscopy reveals areas of intense autofluorescence on the Bgh challenged pen2 plant at a low magnification (8×), indicative of a significantly increased frequency (approx. 30%) of single epidermal cells having undergone an hypersensitive response (HR), as revealed at a higher magnification (100×).

FIG. 3 shows a comparative cytological time course analysis of single Bgh-Col-3/pen2 interaction sites. A significant higher penetration success is invariably ensued by a cell death response on pen2 plants.

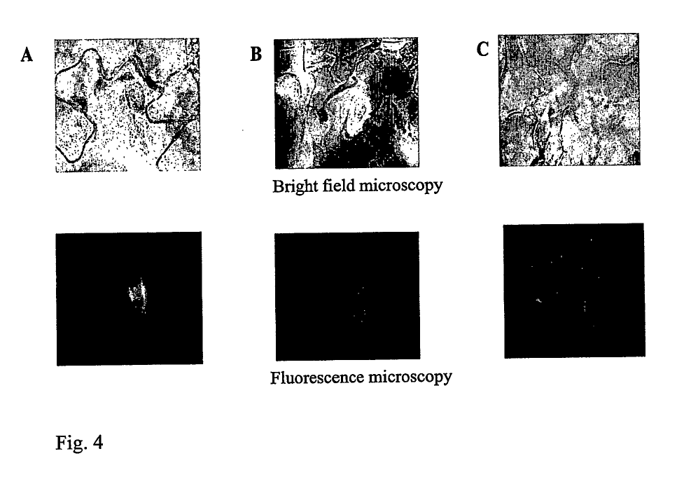

FIG. 4 illustrates the course of pathogen growth (Bgh) on the epidermis of a pen2 mutant leaf.
A. Mature haustorium formation with typical digit-like protrusions in a successfully penetrated pen2 epidermis cell. Fluorescence microscopy reveals an incomplete encapsulation (24 h p. inoc.).
B. Finally all established haustoria become encased in a thick layer of callose and polyphenolic compounds (48 h p. inoc.).
C. Successful penetration invariably leads to a hypersensitive cell death response (72 h p. inoc.).

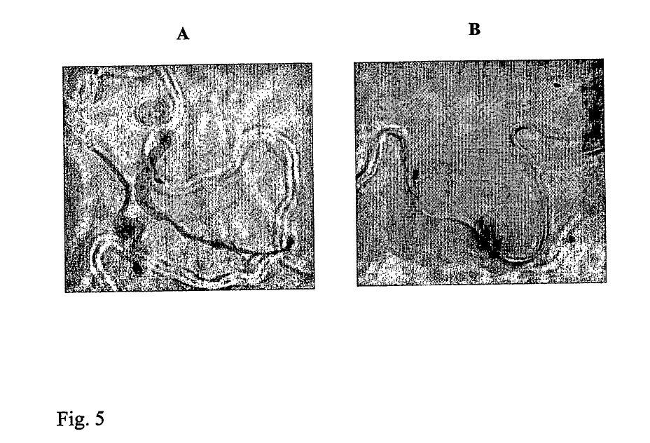

FIG. 5 proves that pen2 mutant plants are penetrated by Phytophthora infestans to a significantly higher extent than corresponding wild-type plants.
A On Col-3 plants the oomycete Phythophthora infestans exhibits only limited superficial growth and almost no successful penetration.
B On pen2 mutant plants a significantly enhanced penetration success is observed. Fungal infection structures become encased in cell wall like material.

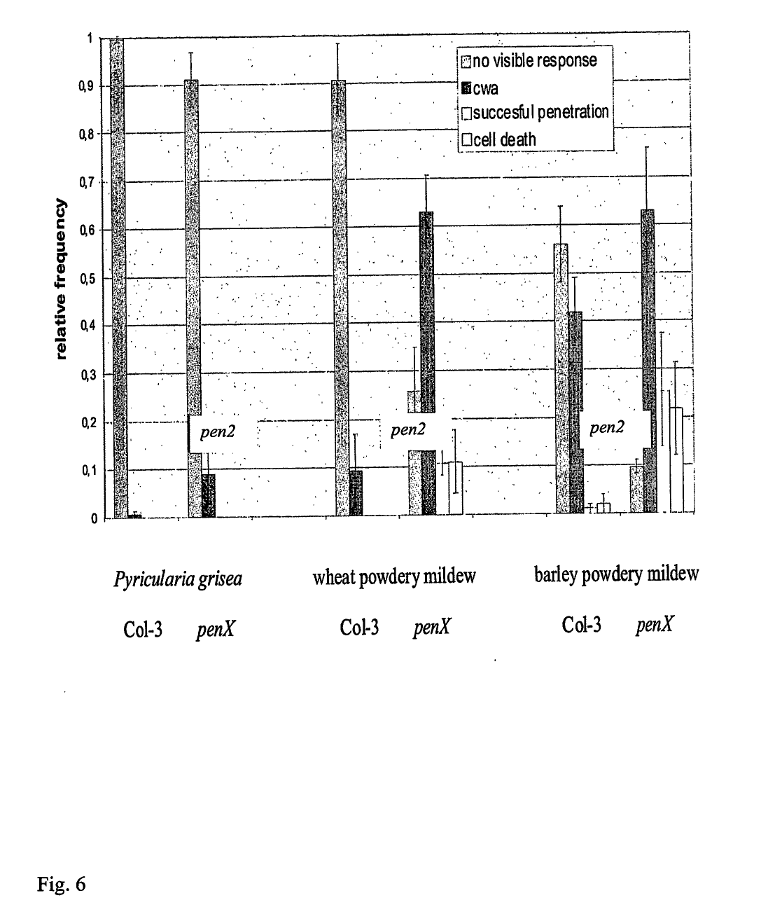

FIG. 6 gives a comparative cytological analysis of Col-3 and pen2 challenged with different avirulent fungal pathogens (72 h p. inoc.). Challenge with the hemibiotrophic rice pathogen P. grisea gives rise to a significantly higher frequency of cell wall remodeling in pen2 plants. Wheat powdery mildew inoculation results in enhanced penetration success and an increased cell death rate in pen2 plants.

FIG. 7 depicts a comparative FTIR spectrum plot showing relative absorbance changes in cell walls of Col-3 and pen2 plants. Significant relative absorbance differences between wildtype Col-3 and mutant pen2 are prominent in the range of carbohydrate and phenolics fingerprints.

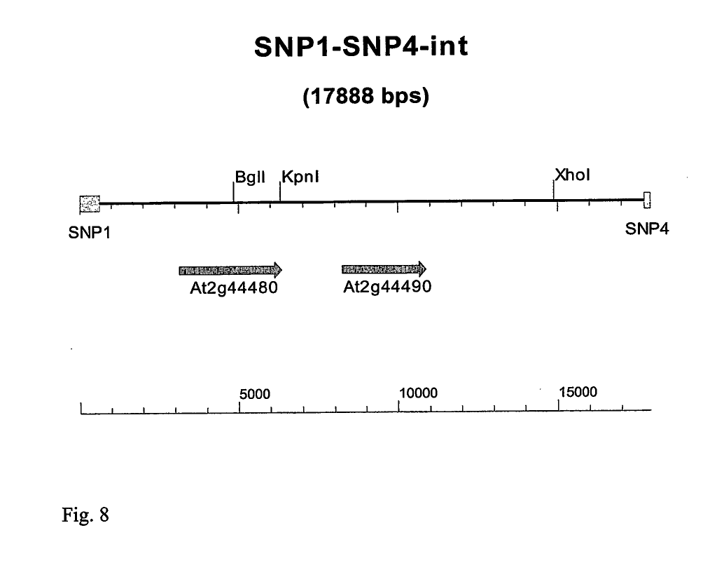

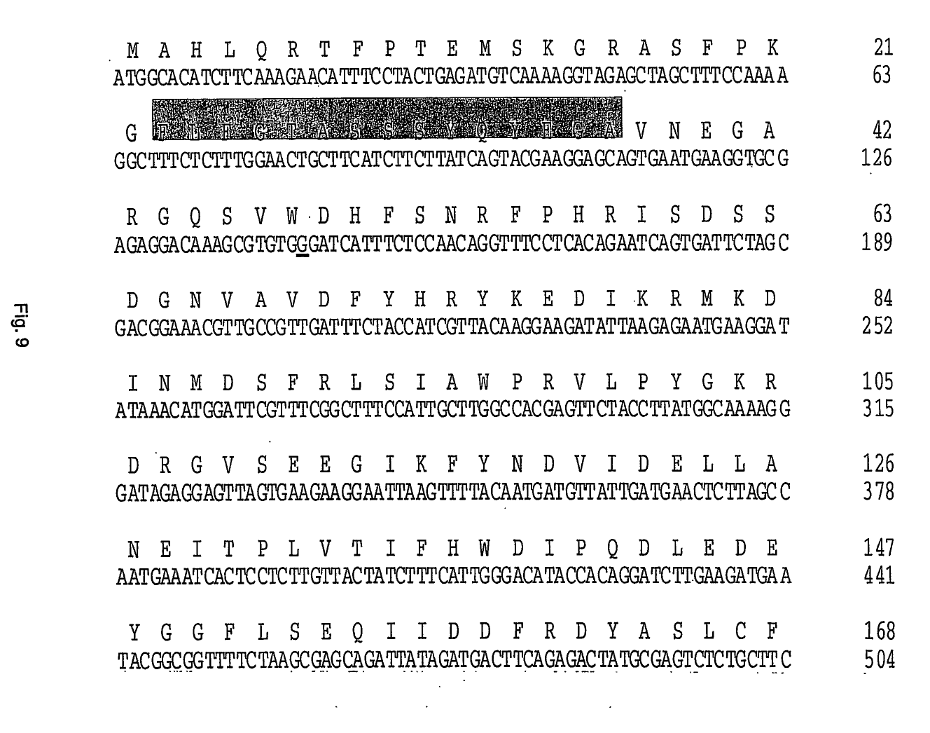

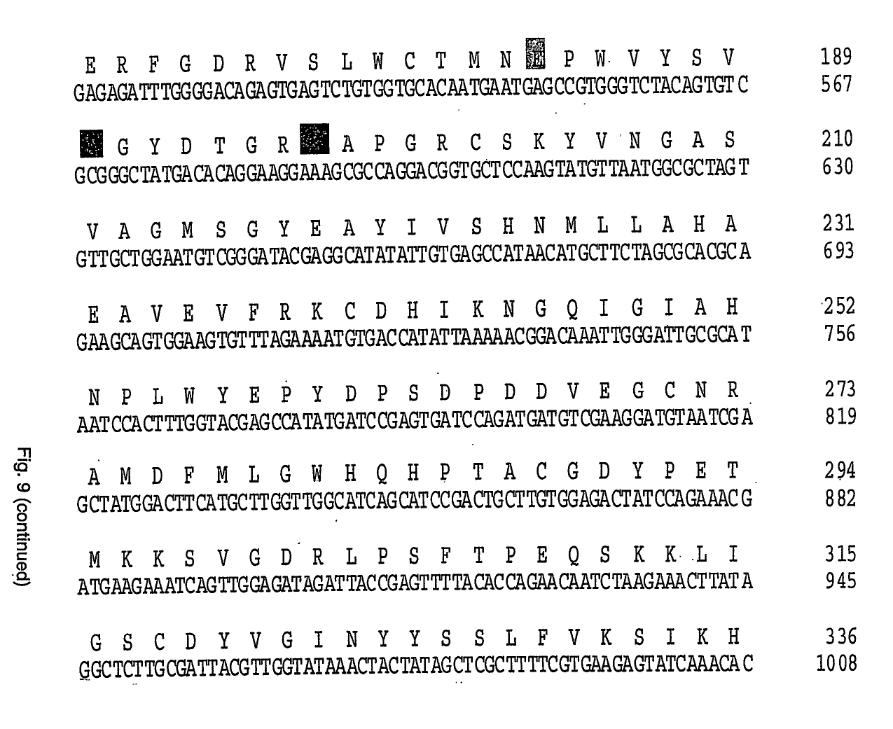

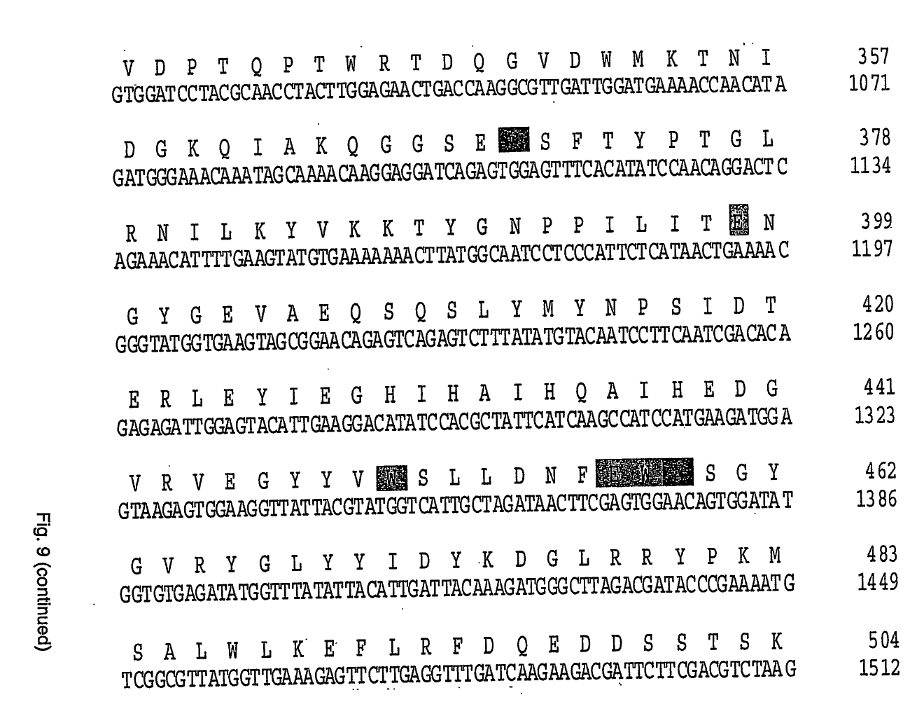

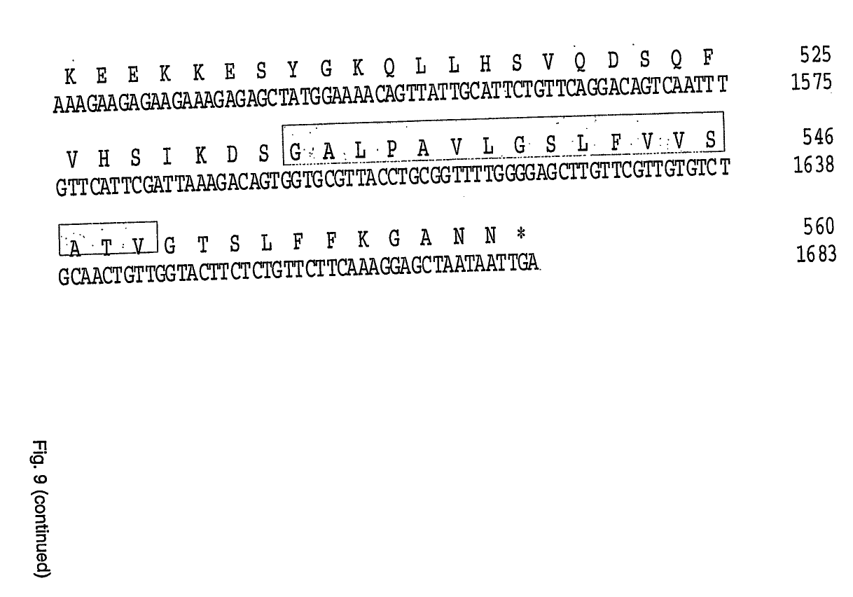

FIG. 8 gives a schematic overview of the chromosomal region harboring the gene encoding pen2. SSLP and CAPS marker assisted map based cloning resulted in an 18 kb fragment of BAC F4I1, flanked by CEREON data based CAPS markers SNP1 and SNP4, containing two predicted genes, both coding for putative family 1 glycohydrolases of which At2g 44490 showed to encode PEN2.

FIG. 9 presents the nucleotide and deduced amino acid sequence of the PEN2 cDNA.

The glycosyl hydrolase family 1 N-terminal signature is boxed in dark gray and the putative C-terminal transmembrane domain boxed in light gray. The G/A144 transition site in the mutant pen2 is underlined. The two catalytic glutamates are boxed in blue, highly conserved residues involved in glucose binding in green and residues involved in determining substrate specificity in red.

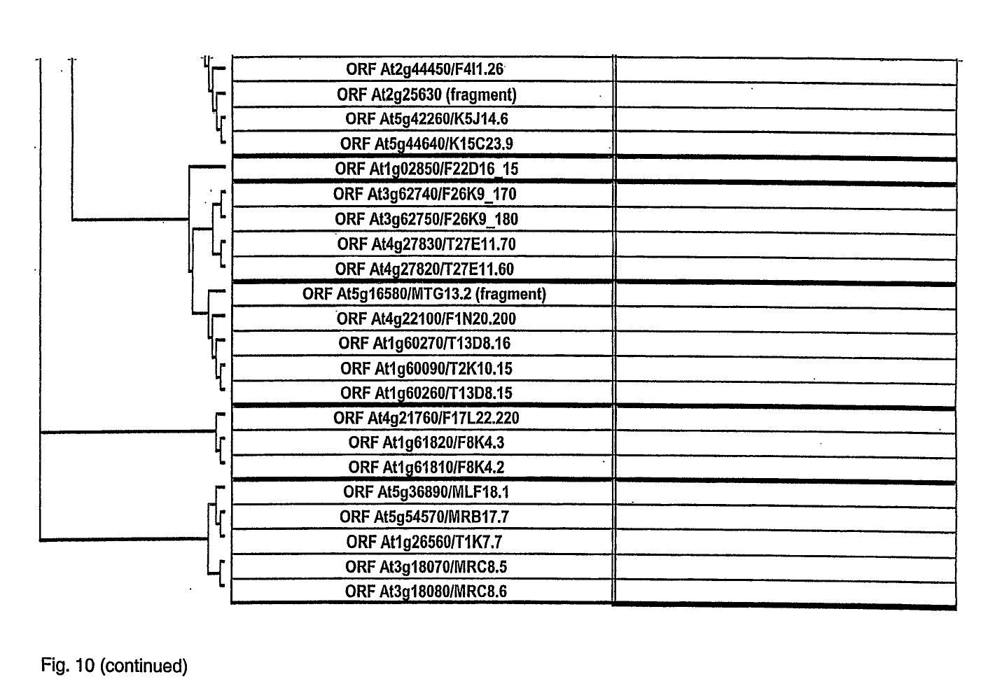

FIG. 10 shows a phylogenetic analysis of all 47 Arabidopsis family 1 glycoside hydrolases. It assigns PEN2 (At2g44490/F4I1.30) to a cluster of 11 proteins, some of which might potentially be involved in plant defense mechanisms and/or senescence.

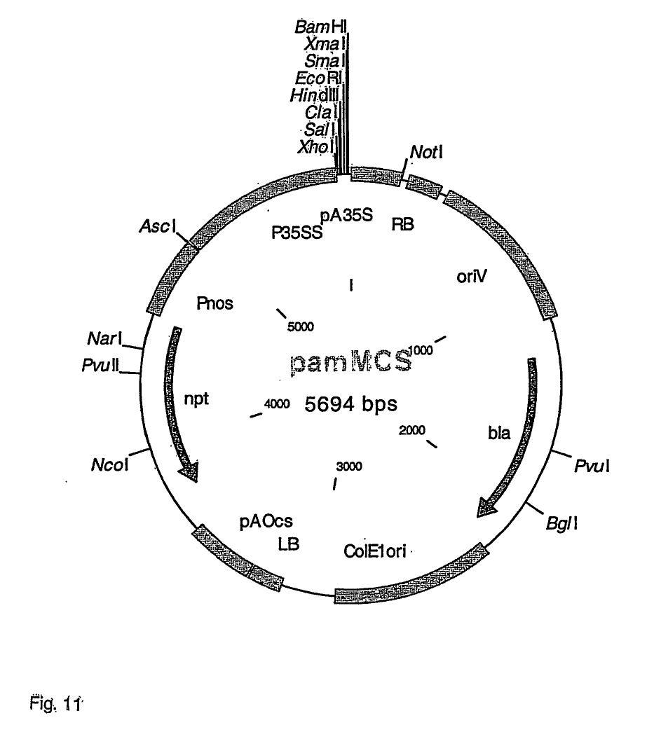

FIG. 11 depicts the plasmid map of the binary plant expression vector pamMCS for use in plant transformation.

FIG. 12 shows a sequence alignment of the PEN2 amino acid sequence with amino acid sequences from related family 1 glycoside hydrolases, showing residues relevant for catalysis and substrate specificity. ATHcab75923, closest Arabidopsis homologue of PEN2; ZMGlu1, maize (Zea mays L.) Glu1; ZMGlu2, maize Glu2; SBDhr1, sorghum (sorghum bicolor) Dhr1; SAMyr, white mustard (sinapis alba) myrosinase. The alignment shows the two catalytic glutamates (*), highly conserved residues involved in glucose binding (+) and residues involved in aglycone binding (#). The N-terminal signature of family 1 β-glycosidases is boxed. Regions of significant sequence similarity are shaded.

The following Examples serve to further illustrate the invention.

In the Examples the following materials and methods were used.

1. Molecular Biological Techniques.

Unless stated otherwise in the Examples, all recombinant DNA techniques are performed according to protocols as described in Sambrook and Russell (2001), Molecular Cloning: A Laboratory Manual, CSH Press, Cold Spring Harbor, N.Y., USA or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, jointly published by BIOS Scientific Publications Ltd (UK) and Blackwell Scientific Publications (UK).

2. Plant and Fungal Material and Growth Conditions

An EMS mutated M2 population of Col-3, gl1 purchased from Lehle Seeds (LEHLE SEEDS 1102 South Industrial Blvd. Suite D Round Rock, Tex. 78681 USA; Cat.-# M2E-01A-05) was used for the mutant screen.

Plants were grown in Percival chambers (AR75L3) at 22° C. and 70% humidity with a 16 h photoperiod. Light intensity was >250 μE/m2 per sec for all experiments.

Blumeria graminis f. sp hordei K1 was maintained on barley (Hordeum vulgare) line Sultan-5.

3. Mutant Screen

3-4 week old Arabidopsis plants were inoculated by placing 16 pots (9×9 cm) each containing 16 plants into a settling tower and tapping six heavily infected barley pots over the top of the tower. After 5 min, the pots were returned to the Percival growth chambers. 5 days post inoculation plants were screened with a Leica stereomicroscope MZFLIII with a GFP1 filter set for aberrant enhanced autofluorescence patterns indicative of enhanced cell death of epidermal cells due to successful fungal ingress.

### EXAMPLE 1

### Cytological Studies on the Interaction Between Arabidopsis and the Non-Host Pathogen Blumeria graminis (Bgh)

As a first step, plant/fungus interaction was analyzed at the cytological level. Bgh spore inoculation of Arabidopsis ecotype Col-3 (gl1) plants showed complete immunity and lack of macroscopically visible plant responses even at late time points after inoculation (seven days after inoculation). Light microscopic analysis revealed successful germination of fungal spores on the leaf surface, efficient differentiation of Bgh germ tubes and appressorium formation, i.e. infection structures known to be critical for penetration of the fungus through the plant cell wall. Unlike compatible interactions on barley leaves, most sporelings failed to successfully penetrate epidermal cell walls at sites of attempted ingress (>98%). At failed penetration sites, host cells remained alive and typically responded to attempted fungal ingress by the formation of round shaped cell wall appositions, containing autofluorescent compounds, directly beneath a fungal appressorium. However, the remaining sporelings (<2%) successfully penetrated the cell wall beneath appressoria, indicated by the presence of haustorial initials within single epidermal cells (FIG. 1). Haustorium maturation ceased in most cases prematurely and haustorium containing host epidermal cells invariably died within 72 hours after inoculation as indicated by a whole cell yellow autofluorescence under UV light irradiation (FIG. 1). This cell death response may reflect activation of plant defense as a consequence of invasive fungal growth. Since both localized cell wall apposition formation and epidermal cell death are also implicated in Bgh defense following attack of its natural host, barley, it may be concluded that the nonhost Arabidopsis is capable to trigger similar cellular defense reactions. It was speculated that the low incidence of fungal cell wall penetration into Arabidopsis epidermal cells and the coincident formation of cell wall appositions at failed penetration sites might reflect the presence of effective (prehaustorial) nonhost resistance in Col-3 (gl1) wild type plants, preventing a switch from surface to invasive fungal growth. Taken together, non-host resistance of Arabidopsis to Bgh appears to be a predominantly prehaustorial, active defense response manifested at the level of cell wall prenetration.

### EXAMPLE 2

### Isolation of Arabidopsis Mutants with Aberrant Infection Phenotypes to Bgh

The information obtained from failed infection attempts of fungal sporelings on wild type Arabidopsis (see Example 1) could be used to devise a fluorescence-based screening procedure that uses plant autofluorescence as read out to identify Arabidopsis mutants exhibiting aberrant infection phenotypes. One could reason that genetic defects in prehaustorial nonhost defense would lead to an increased haustorium index. Since intracellular growth of the nonhost fungus triggers host cell death (see above), enhanced epidermal cell death rates in prehaustorial defense mutants may be expected. Autofluorescence-associated cell death of epidermal cells can be easily detected by irradiating spore-inoculated Arabidopsis plants with UV-light and examining leaves using a fluorescence stereomicroscope. Col-3 (gl1) wild type plants show even after high density spore inoculation only occasional yellow autofluorescence in the epidermal cell layer at five days after inoculation (FIG. 2). This stereomicroscope-based assay was then used to screen 26,260 M2 plants of an EMS mutagenized Col-3 (gl1) population for mutants exhibiting a higher index of epidermal whole cell autofluorescence compared to wild type plants. Amongst several mutant candidates, a heritable mutant could be isolated which has been designated pen2 (FIG. 2). Quantitative cytological analysis of single interaction sites revealed in pen2 mutants about 15-fold enhanced penetration frequencies compared to Col-3 plants, thereby increasing the haustorial index from 2 to approximately 30% (FIG. 3). The cytological analysis frequently showed fully differentiated Bgh haustoria within penetrated pen2 epidermal cells 24 h after spore inoculation, which became normally encapsulated by cell wall-like material at ˜48 h. Similar to Col-3 wild type plants, a cell death response ensued in haustorium containing pen2 epidermal cells between 24 and 48 h post inoculation (FIGS. 3 and 4).

### EXAMPLE 3

### pen2 Plants Reveal Compromised Nonhost Resistance to Different Fungal Pathogens

To find out whether pen2 plants were compromised in defense to other nonhost pathogens, wild type and pen2 plants were challenged with Pyricularia grisea, Blumeria graminis f sp tritici (Bgt; wheat powdery mildew), and Phytophthora infestans. In either of the tested plant fungus interactions, evidence was found for compromised nonhost resistance in pen2 plants (FIGS. 5 and 6). For example, challenge with Bgt or P. infestans showed at interaction sites on pen2 plants strikingly enhanced penetration frequencies that are accompanied by elevated cell death frequencies. This is remarkable because P. infestans is an oomycete and Bgt and Bgh are ascomycete fungi, indicating impaired nonhost resistance in pen2 to different classes of pathogens. Interestingly, Pyricularia grisea failed to trigger any microscopically visible cellular responses in Col-3 plants despite its ability to differentiate melanized appressoria for cell wall penetration on the leaf surface. In pen2 plants, in about 10% of the interaction sites, cell wall apposition formation was observed which is indistinguishable from the round shaped wall appositions seen in response to attempted Bgh penetration. Likewise, the frequency of wall apposition formation in response to Bgt challenge is dramatically enhanced in pen2 plants compared to Col-3 (63% and 9% respectively). Collectively, these data point to a dual role of PEN2 in static and inducible defense to various non-host fungal pathogens. Dependent on the particular non-host pathogen, lack of PEN2 can enhance the frequency of inducible cellular defense (wall apposition formation) as consequence of an impaired static barrier and/or increase the incidence of failed inducible defense coincident with host cell wall penetration.

### EXAMPLE 4

### FTIR Analysis Suggests a Role for PEN2 in Cell Wall Architecture

In view of the enhanced cell wall penetration frequencies of several non-host fungal pathogens on pen2 plants, it was interesting to investigate a potential role of PEN2 in cell wall architecture. Fourier transformed infrared spectroscopy (FTIR) of non-infected wild type and pen2 leaf samples (FIG. 7) was performed. FTIR is a rapid and non-invasive spectroscopic method that can quantitatively detect a range of chemical groups and bonds in fixed plant specimens. The difference FTIR spectrum between wild type and pen2 leaves indicates significant constitutive changes in cell wall architecture of pen2 plants. This is consistent with the interpretation that pen2 plants are impaired in static pathogen defense that may be critical during attempted host cell wall penetration.

### EXAMPLE 5

### Map-Based Isolation of PEN2

An F2 mapping population was generated by crosses between pen2 and Landsberg (La) wild-type plants. Bgh inoculation experiments of individual F2 plants showed that pen2 segregated as monogenic recessive trait. DNA marker analysis of bulked F2 segregants suggested that pen2 maps close to the telomere on the short arm of chromosome 2. By using CAPS and SSLP-type DNA markers, pen2 could be located in a 7.4 cM target interval bordered by markers F18O19 and F4I18 (see FIG. 8). Molecular analysis of 864 F2 individuals from the mapping population identified a total of 24 recombinants within the 7.4 cM target interval. Scoring of infection phenotypes of the 24 recombinant plants upon Bgh challenge enabled further delimitation of pen2 to an interval of approximately 18 kb harboring two predicted ORFs (At2g44480 and At2g44490; see GenBank/EMBL database entry No. AC004521). Both deduced gene products show significant homology to family 1 glycosyl hydrolases. RT-PCR of leaf RNA samples resulted in cDNA transcripts for At2g44490 only. Sequencing of pen2 derived At2g44490 cDNAs (and genomic sequences) showed in comparison to wild type cDNAs a single G to A transition at position 144, generating a stop codon and a predicted truncated peptide of 47 aa. No sequence polymorphism was found upon comparison of At2g44480 genomic sequences derived from wild type and pen2 mutant DNA. Collectively, these data made it possible to identify the pen2 gene (At2g44490) within the genetically and physically delimited target interval. The RT-PCR derived PEN2 cDNA sequence and the deduced PEN2 protein sequence are shown in FIG. 9.

Glycoside hydrolases catalyze the hydrolysis of glycosidic bonds in oligosaccharides, polysaccharides and conjugates between glucosides and a non-carbohydrate moiety. They occur in all living organisms and are classified into 82 families (http://afmb.cnrs-mrs.fr/˜cazy/CAZY/; Henrissat, Biochem. J. 316 (1996), 695-696). Due to the presence of a specific motif (family 1 N-terminal signature; see FIG. 9), PEN2 is assigned to family 1 glycoside hydrolases. Known enzymatic activities of family 1 enzymes range from β-glucosidase, β-galactosidase, 6-phopho-β-galactosidase, 6-phospho-β-glucosidase, lactase-phlorizin hydrolase, β-mannosidase, and myrosinase activities. A total of 282 different family 1 glycoside hydrolase proteins are at present classified in this group, including 106 plant sequences of which 47 are Arabidopsis thaliana sequences. PEN2 shows highest homology to β-glucosidases that constitute a major group among family 1 glycoside hydrolases. These enzymes hydrolyze either O-linked or S-linked β-glucosidic bonds. Substrates for these proteins are extraordinary diverse and include hormone glucosides, flavonol glucosides, cyanogenic glucosides, and glucosinolates. The aglycon product of the hydrolyzed substrates can serve a multitude of functions including growth and development (Selmar et al., 1987; Brzobohaty et al., 1993; Dietz et al., 2000), cell wall catabolism (Leah et al., 1995; Gerardi et al., 2001), lignification (Dharmawardhana et al., 1995 and 1999), and defense (Zheng and Poulton, 1995; Rsak et al., 2000).

A phylogenetic analysis using Clustal W algorithm of all known 47 Arabidopsis β-glucosidases identifies several major branches and some outgroups (FIG. 10). Interestingly, PEN2 outgroups together with another family member (T2O9.100) in a major branch comprising a total of 11 β-glucosidases (FIG. 10). The fact that the closest relative shares only 55.2% sequence identity (and 70.5% similarity) with PEN2 underlines the unusual sequence divergence of β-glucosidases within a species.

### EXAMPLE 6

### Overexpressing PEN2 in Transgenic Arabidopsis Plants

Transgenic Arabidopsis thaliana PEN2 overexpressing lines are generated by fusing the 35S promoter upstream of the PEN2 coding region, followed by pA35S terminator sequences. For this purpose the binary plasmid DNA vector pamMCS is used (FIG. 11).

Agrobacterium-mediated flower dip transformation and kanamycin selection is used to identify transgenic lines (Clough, Plant Journal. 16 (1998), 735-743). Immunoblots from tissue extracts of individual transgenic lines are probed with a PEN2 specific antiserum to select overexpressing PEN2 lines. The transgenic plants will be tested for enhanced disease resistance to several pathogens, including Blumeria graminis, Erysiphe cichoracearum, Peronospora parasitica, Pseudomonas syringae, Fusarium oxysporum, Botrytis cinerea, and Phytophthora infestans. Similar experiments will be conducted to generate and isolate PEN2 overexpressing lines in closely related plant species (e.g. Brassica) or more distantly related species including tomato and potato and will be tested for enhanced disease resistant phenotypes.

### CITED LITERATURE

Amano(Hirata), K. 1986. Host range and georaphic distribution of the powdery mildew fungi. Japan Scientific Societies Press, Tokyo, Japan.
Brzobohaty Bretislav. Moore Ian. Kristoffersen Peter. Bako Laszlo. Campos Narciso. Schell Jeff. Palme Klaus [a]. Release of active cytokinin by a beta-glucosidase localized to the maize root meristem. Science (Washington, D.C.). 262(5136). 1993. 1051-1054
Dangl Jeffery L [a]. Jones Jonathan D G. Plant pathogens and integrated defence responses to infection. Nature (London). 411(6839). 2001. 826-833.
Dietz Karl-Josef [a]. Sauter Angela. Wichert Kathrin. Messdaghi David. Hartung Wolfram. Extracellular beta-glucosidase activity in barley involved in the hydrolysis of ABA glucose conjugate in leaves. Journal of Experimental Botany. [print.] 51(346). May, 2000. 937-944
Dharmawardhana D Palitha. Ellis Brian E. Carlson John E [a]. A beta-Glucosidase from Lodgepole Pine Xylem Specific for the Lignin Precursor Coniferin. Plant Physiology (Rockville). 107(2). 1995. 331-339
Dharmawardhana D Palitha. Ellis Brian E [a]. Carlson John E. cDNA cloning and heterologous expression of coniferin beta-glucosidase. Plant Molecular Biology. 40(2). May, 1999. 365-372
Fluhr R. Sentinels of disease. Plant resistance genes. Plant Physiology 127 December 2001. 1367-1374
Gerardi C. Blando F. Santino A. Zacheo G. Purification and characterisation of a β-glucosidase abundantly expressed in ripe sweet cherry (Prunus avium L.) fruit. Plant Science. 160. 795-805. 2001
Heath Michele C [a]. Nonhost resistance and nonspecific plant defenses. Current Opinion in Plant Biology. 3(4). August, 2000. 315-319
Kamoun Sophien [a]. Van West Pieter. Vleshouwers Vivianne G A A. De Groot Koen E. Govers Francine. Resistance of Nicotiana benthamiana to Phytophthora infestans is mediated by the recognition of the elicitor protein INF1. Plant Cell. 10(9). September, 1998. 1413-1425.
Leah R. Kigel J. Svendsen I. Mundy J. Downstream processing of cell wall polysaccharides of barley seed involves a beta-glucosidase. Journal of the Institute of Brewing. 101(3). 1995. 154
Rask Lars [a]. Andreasson Erik. Ekbom Barbara. Eriksson Susanna. Pontoppidan Bo. Meijer Johan. Myrosinase: Gene family evolution and herbivore defense in Brassicaceae. Plant Molecular Biology. 42(1). January, 2000. 93-113
Selmar D. Lieberei R. Biehl B. Voigt J. Hevea Linamarase a nonspecific beta glycosidase. Plant Physiology (Bethesda) 83 (3). 1987. 557-563
Zheng Liansheng. Poulton Jonathan E [a]. Temporal and spatial expression of amygdalin hydrolase and (R)-(+)-mandelonitrile lyase in black cherry seeds. Plant Physiology (Rockville). 109(1). 1995. 31-39

1. A recombinant nucleic acid molecule comprising a polynucleotide selected from the group consisting of
(a) polynucleotides comprising a nucleotide sequence encoding a polypeptide with the amino acid sequence of SEQ ID NO:2;
(b) polynucleotides comprising the nucleotide sequence shown in SEQ ID NO:1;
(c) polynucleotides comprising a nucleotide sequence encoding a fragment of the polypeptide encoded by a polynucleotide of (a) or (b), wherein said nucleotide sequence encodes a protein having β-glucosidase activity;
(d) polynucleotides comprising a nucleotide sequence which encodes a polypeptide having a sequence identity of at least 50% to an amino acid sequence encoded by the polynucleotide of (a) or (b), wherein said polypeptide has β-glucosidase activity; and
(e) polynucleotides comprising a nucleotide sequence that deviates from the nucleotide sequence defined in (d) by the degeneracy of the genetic code;
and a promoter operatively linked to said polynucleotide, said promoter being heterologous with respect to the polynucleotide.

2. The recombinant nucleic acid molecule of claim 1 further comprising additional expression control sequences operably linked to said polynucleotide.

3. A vector comprising the polynucleotide defined in claim 1 or the recombinant nucleic acid molecule of claim 1.

4. The vector of claim 3 further comprising expression control sequences operably linked to said polynucleotide.

5. A method for producing genetically engineered host cells comprising introducing into a host cell the polynucleotide defined in claim 1, the recombinant nucleic acid molecule of claim 1 or a vector comprising the polynucleotide or the recombinant nucleic acid molecule and further comprising an expression control sequence operably linked to said polynucleotide.

6. A host cell which is obtained by the method of claim 5.

7. The host cell of claim 6 which is a bacterial, yeast, fungus, plant or animal cell.

8. A method for the production of a polypeptide encoded by a polynucleotide defined in claim 1 in which a host cell is obtained by introducing into the host cell the polynucleotide defined in claim 1, the recombinant nucleic acid molecule of claim 1 or a vector comprising the polynucleotide or the recombinant nucleic acid molecule and further comprising an expression control sequence operably linked to said polynucleotide and wherein the host cell is cultivated under conditions allowing for the expression of the polypeptide and in which the polypeptide is isolated from the cells and/or the culture medium.

9. A polypeptide encoded by the polynucleotide defined in claim 1.

10. An antibody specifically recognizing the polypeptide of claim 9.

11. A method for producing a transgenic plant comprising the steps of
(a) introducing the polynucleotide defined in claim 1, the recombinant nucleic acid molecule of claim 1 or a vector comprising the polynucleotide or the recombinant nucleic acid molecule into the genome of a plant cell; and
(b) regenerating the cell of (a) to a transgenic plant.

12. A transgenic plant or plant tissue comprising the plant cells of claim 7.

13. A transgenic plant which shows an increased activity of the polypeptide encoded by the polynucleotide defined in claim 1 compared to a corresponding wild-type plant.

14. The transgenic plant of claim 12 which, upon an increased activity of the protein encoded by the polynucleotide, shows an increased resistance against a plant pathogen to which a corresponding wild-type plant is susceptible.

15. Propagation material or harvestable parts of a transgenic plant comprising plant cells of claim 7.

16. A method for conferring pathogen resistance or increased pathogen resistance to a plant comprising the step of providing a transgenic plant in which the activity of the polypeptide encoded by the polynucleotide defined in claim 1 is increased.

17. A method for identifying a compound that is hydrolyzed by a polypeptide encoded by the polynucleotide defined in claim 1 comprising the steps of
(a) contacting a candidate compound with said polypeptide under conditions where said polypeptide is active; and
(b) determining whether said candidate compound is hydrolyzed by said polypeptide.

18. A method for identifying a compound that is hydrolyzed by a polypeptide encoded by the polynucleotide defined in claim 1 comprising the steps of
(a) providing a 3-dimensional structure model of said polypeptide; and
(b) determining the structure of a substrate that fits into the 3-dimensional structure model of (a).

19. A method for identifying a compound that is hydrolyzed by a polypeptide encoded by the polynucleotide defined in claim 1 comprising the steps of
(a) providing a mutant protein of said polypeptide the catalytic activity of which is abolished without losing substrate binding activity;
(b) contacting a candidate compound with said mutant protein; and
(c) determining whether the candidate compound is bound by said mutant protein.

20. The method of claim 17 furthermore comprising the step of determining whether the identified compound or a hydrolysis product thereof is capable of inducing or enhancing a defence response against a pathogen in a plant.

21. A method for preparing a plant protection composition comprising the steps of the method of claim 17 and furthermore the step of formulating the identified compound or a hydrolysis product thereof in a form suitable for administering to plants.

22. A compound, a hydrolysis product thereof or a plant protection composition identified or obtained by the method claim 16.

23. A kit comprising the polynucleotide defined in claim 1, the recombinant nucleic acid molecule of claim 1, or a vector 4 comprising the polynucleotide or the recombinant nucleic acid molecule, a polypeptide encoded by the polynucleotide defined in claim 1, or an antibody recognizing the polypeptide.

24. (canceled)

25. (canceled)

26. (canceled)


SEQUENCE LISTING (9 sequences)

SEQ ID NO: 1 - DNA, 1683 bp, Arabidopsis thaliana
  CDS (1)..(1680)
atggcacatcttcaaagaacatttcctactgagatgtcaaaaggtagagctagctttccaaaaggctttctctttggaactgcttcatcttcttatcagtacgaaggagcagtgaatgaaggtgcgagaggacaaagcgtgtgggatcatttctccaacaggtttcctcacagaatcagtgattctagcgacggaaacgttgccgttgatttctaccatcgttacaaggaagatattaagagaatgaaggatataaacatggattcgtttcggctttccattgcttggccacgagttctaccttatggcaaaagggatagaggagttagtgaagaaggaattaagttttacaatgatgttattgatgaactcttagccaatgaaatcactcctcttgttactatctttcattgggacataccacaggatcttgaagatgaatacggcggttttctaagcgagcagattatagatgacttcagagactatgcgagtctctgcttcgagagatttggggacagagtgagtctgtggtgcacaatgaatgagccgtgggtctacagtgtcgcgggctatgacacaggaaggaaagcgccaggacggtgctccaagtatgttaatggcgctagtgttgctggaatgtcgggatacgaggcatatattgtgagccataacatgcttctagcgcacgcagaagcagtggaagtgtttagaaaatgtgaccatattaaaaacggacaaattgggattgcgcataatccactttggtacgagccatatgatccgagtgatccagatgatgtcgaaggatgtaatcgagctatggacttcatgcttggttggcatcagcatccgactgcttgtggagactatccagaaacgatgaagaaatcagttggagatagattaccgagttttacaccagaacaatctaagaaacttataggctcttgcgattacgttggtataaactactatagctcgcttttcgtgaagagtatcaaacacgtggatcctacgcaacctacttggagaactgaccaaggcgttgattggatgaaaaccaacatagatgggaaacaaatagcaaaacaaggaggatcagagtggagtttcacatatccaacaggactcagaaacattttgaagtatgtgaaaaaaacttatggcaatcctcccattctcataactgaaaacgggtatggtgaagtagcggaacagagtcagagtctttatatgtacaatccttcaatcgacacagagagattggagtacattgaaggacatatccacgctattcatcaagccatccatgaagatggagtaagagtggaaggttattacgtatggtcattgctagataacttcgagtggaacagtggatatggtgtgagatatggtttatattacattgattacaaagatgggcttagacgatacccgaaaatgtcggcgttatggttgaaagagttcttgaggtttgatcaagaagacgattcttcgacgtctaagaaagaagagaagaaagagagctatggaaaacagttattgcattctgttcaggacagtcaatttgttcattcgattaaagacagtggtgcgttacctgcggttttggggagcttgttcgttgtgtctgcaactgttggtacttctctgttcttcaaaggagctaataattga

SEQ ID NO: 2 - protein, 560 aa, Arabidopsis thaliana
MAHLQRTFPTEMSKGRASFPKGFLFGTASSSYQYEGAVNEGARGQSVWDHFSNRFPHRISDSSDGNVAVDFYHRYKEDIKRMKDINMDSFRLSIAWPRVLPYGKRDRGVSEEGIKFYNDVIDELLANEITPLVTIFHWDIPQDLEDEYGGFLSEQIIDDFRDYASLCFERFGDRVSLWCTMNEPWVYSVAGYDTGRKAPGRCSKYVNGASVAGMSGYEAYIVSHNMLLAHAEAVEVFRKCDHIKNGQIGIAHNPLWYEPYDPSDPDDVEGCNRAMDFMLGWHQHPTACGDYPETMKKSVGDRLPSFTPEQSKKLIGSCDYVGINYYSSLFVKSIKHVDPTQPTWRTDQGVDWMKTNIDGKQIAKQGGSEWSFTYPTGLRNILKYVKKTYGNPPILITENGYGEVAEQSQSLYMYNPSIDTERLEYIEGHIHAIHQAIHEDGVRVEGYYVWSLLDNFEWNSGYGVRYGLYYIDYKDGLRRYPKMSALWLKEFLRFDQEDDSSTSKKEEKKESYGKQLLHSVQDSQFVHSIKDSGALPAVLGSLFVVSATVGTSLFFKGANN

SEQ ID NO: 3 - protein, 5 aa, Artificial Sequence
  Description of Artificial Sequence Synthetic peptide
TFNEP

SEQ ID NO: 4 - protein, 5 aa, Artificial Sequence
  Description of Artificial Sequence Synthetic peptide
  MOD_RES (1): Ile or Val
XTENG

SEQ ID NO: 5 - protein, 534 aa, Arabidopsis thaliana
MYSKKNSFGRSDFPEGFLFGTASSAYQYEGARNEAPRGESVWDTFVRKYPERNCYSNADQAIEFYNHYKDDIQRMKDINMDAFRFSISWPRIFPLQKKSKQVNKEGIQFYNDLIDELLANGITPLATLFHWDTPQALEDEYSGFLSEEAVDDFKDFAALCFEEFGDRVKLWVTLNEPWVYSIGGYDTGRKAPGRASKYMNEAAVAGESGLEVYTVSHNLLLAHAEAVEVFRNNPKCKDGKIGIAHCPVWFEPYDSNCPKDIEACERAMEFMFGWHMDPTVYGDYPAVMKKSIGKRLPSFTAAQSKKLRGSFDFVGVNYYSAFYVKNIDEVNHDKPNWRSDARIEWRKENNAGQTLGVRGGSEWDFLYPQGLRKFLNYAKNKYESPKFMITENGHCDIDYEKKPKLSNLMDLQRTEYHKKHLQSIQQAIQEDGVVVEGYFAWSLLDNCEWNAGYGVRYGLFYVDYNNGLKRFPKMSAMWFKEFLKREEEIEDSEEEEYVLKSTMNKKRFLLATGSSASCFIPKMSESSKALELLF

SEQ ID NO: 6 - protein, 566 aa, Zea mays
MAPLLAAAMNHAAAHPGLRSHLVGPNNESFSRHHLPSSSPQSSKRRCNLSFTTRSARVGSQNGVQMLSPSEIPQRDWFPSDFTFGAATSAYQIEGAWNEDGKGESNWDHFCHNHPERILDGSNSDIGANSYHMYKTDVRLLKEMGMDAYRFSISWPRILPKGTKEGGINPDGIKYYRNLINLLLENGIEPYVTIFHWDVPQALEEKYGGFLDKSHKSIVEDYTYFAKVCFDNFGDKVKNWLTFNEPQTFTSFSYGTGVFAPGRCSPQLDCAYPTGNSLVEPYTAGHNILLAHAEAVDLYNKHYKRDDTRIGLAFDVMGRVPYGTSFLDKQAEERSWDINLGWFLEPVVRGDYPFSMRSLARERLPFFKDEQKEKLAGSYNMLGLNYYTSRFSKNIDISPNYSPVLNTDDAYASQEVNGPDGKPIGPPMGNPWIYMYPEGLKDLLMIMKNKYGNPPIYITENGIGDVDTKETPLPMEAALNDYKRLDYIQRHIATLKESIDLGSNVQGYFAWSLLDNFEWFAGFTERYGIVYVDRNNNCTRYMKESAKWLKEFNTAKKPSKKILTPA

SEQ ID NO: 7 - protein, 563 aa, Zea mays
MAPLLAAAMNHAAHPVLRSHLGPNNESFSRHHLSSSPQSSKRRFNLSFTPRSARVGNQNGVQLLSPSEIPRRDWFPSDRIFGAATSAYQIEGAWNEDGKGESNWDHFCHNFPERIMDGSNADIGANSYHMYKTDVRLLKEMGMDAYRFSISWPRILPKGTVEGGINQDGIDYYKRLINLLLENGIEPYVTIFHWDVPQALEEKYGGFLDKTQKRIVNDYKNFAKVCFDNFGDKVKNWLTFNEPQTFTSFSYGTGVFAPGRCSPGLDCAIPTGNSLVEPYIAGHNILLAHAEAVDLYNKYYKGENGRIGLAFDVMGRVPYGTSFLDEQAKERSMDINLGWFLEPVVRGDYPFSMRSLARERLPFFSDKQQEKLVGSYNMLGINYYTSIFSKHIDISPKYSPVLNTDDAYASQETYGPDGKPIGPPMGNPWIYLYPEGLKDILMIMKNKYGNPPIYITENGIGDVDTKEKPLPMEAALNDYKRLDYIQRHISTLKESIDLGANVHGYFAWSLLDNFEWYAGYTERYGIVYVDRKNNYTRYMKESAKWLKEFNTAKKPSKKIITPA

SEQ ID NO: 8 - protein, 565 aa, Sorghum bicolor
MALLLASAINHTAHPAGLRSHPNNESFSRHHLCSSPQNISKRRSNLSFRPRAQTISSESAGIHRLSPWEIPRRDWFPPSFLFGAATSAYQIEGAWNEDGKGPSTWDHFCHNFPEWIVDRSNGDVAADSYHMYAEDVRLLKEMGMDAYRFSISWPRILPKGTLAGGINEKGVEYYNKLIDLLLENGIEPYITIFHWDTPQALVDAYGGFLDEEDYKDYTDFAKVCFEKFGKTVKNWLTFNEPETFCSVSYGTGVLAPGRCSPGVSCAVPTGNSLSEPYIVAHNLLRAHAETVDIYNKYHKGADGRIGLALNVFGRVPYTNTFLDQQAQERSMDKCLGWFLEPVVRGDYPFSMRVSARDRVPYFKEKEQEKLVGSYDMIGINYYTSTFSKHIDLSPNNSPVLNTDDAYASQETKGPDQNAIGPPTGNAWINMYPKGLHDILMTMKNKYGNPPMYITENGMGDIDKGDLPKPVALEDHTRLDYIGRHLSVLKQSIDLGADVRGYFAWSLLDNFEWSSGYTERFGIVYVDRENGCERTMKRSARWLGEFNGAAKKVENNKILTPAGQLN

SEQ ID NO: 9 - protein, 544 aa, Sinapis alba
MKLLHGLALVFLLAAASCKADEEITCEENEPFTCSNTDILSSKNFGKDFIFGVASSAYQIEGGRGRGVNVWDGFSHRYPEKSGSDLKNGDTSCESYTRWKKDVEIMGELNATGYRFSFAWSRIVPKQKVSRQVDQAGLDYYHNLIDALLEKNITPFVTLFHWDLPQTLQDEYEGFLDRQIIQDFKDYADLCFKEFGGKVKNWITINQLYTVPTRGYALGTDAPGRCSPKVDTKQRCYGGNSSTEPYIVAHNQLLAHAAIVDLYRTNYAFQNGKIGPVMITRWFLPYDESDPACIEAAERMNQFFHGWYMEPLTKGRYPDIMRQIVGSRLPNFTEAEAELVAGSYDFLGLNYYVTQYAKPKPNPYPSETHTALMDAGVDLTFNNSRQEYPGPVFAEDANSYYYPKGIYYVMDYFKTKYNNPLIYITENGISTPGSESRCEAIADYKRINYLCSHLCFLRKVIREKGVNIRGYFAWALGDNYEFCKQFTVRFGLSYVNWDDLDDRNLKESGKWYQRFINGTAKNPVKQDFLRSSLSSQSQKKRLAC